(19) United States
(12) Patent Application Publication
(10) Pub. No.: US 2003/0232817 A1
(43) Pub. Date: Dec. 18, 2003

(54) Small molecules useful for the treatment of inflammatory disease

(75) Inventor: Roman Wolfgang Fleck, Greenwich, CT (US)

Correspondence Address: BOEHRINGER INGELHEIM CORPORATION, 900 RIDGEBURY ROAD, P O BOX 368, RIDGEFIELD, CT, 06877, US

(73) Assignee: Boehringer Ingelheim Pharmaceuticals, Inc., Ridgefield, CT (US)

(21) Appl. No.: 10/372,333

(22) Filed: Feb. 24, 2003

Related U.S. Application Data
(60) Provisional application No. 60/384,068, filed on May 29, 2002.

(51) Int. Cl. C07D 487/02; A61K 31/541; A61K 31/5377; A61K 31/53; A61K 31/52; A61K 31/517; A61K 31/502; A61K 31/501; A61K 31/497; A61K 31/4188; A61K 31/4745; A61K 31/4439

(52) U.S. Cl. 514/228.2; 514/234.2; 514/248; 514/249; 514/256; 514/241; 514/254.06; 514/255.05; 514/263.2; 514/266.23; 514/338; 514/300; 514/251; 514/365; 514/252.06; 514/372; 514/374; 514/383; 514/387; 544/60; 544/139; 544/209; 544/277; 544/238; 544/237; 544/284; 544/333; 544/405; 546/122; 546/273.1; 548/181; 548/215; 548/302.7

(57) ABSTRACT

Compounds of the formula I

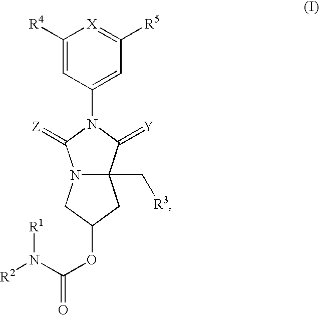

which are useful for treating or preventing inflammatory and immune cell-mediated diseases.

### RELATED APPLICATIONS

Benefit of U.S. Provisional Application Serial No. 60/384,068, filed on May 29, 2002 is hereby claimed.

### BACKGROUND OF THE INVENTION

1. Technical Field

The present invention relates generally to a series of novel small molecules, their synthesis and their use in the treatment of inflammatory disease.

2. Background Information

Research spanning the last decade has helped to elucidate the molecular events attending cell-cell interactions in the body, especially those events involved in the movement and activation of cells in the immune system. See generally, Springer, T. Nature, 1990, 346, 425-434. Cell surface proteins, and especially the Cellular Adhesion Molecules (“CAMs”) and “Leukointegrins”, including LFA-1, MAC-1 and gp150.95 (referred to in WHO nomenclature as CD18/CD11a, CD18/CD11b, and CD18/CD11c, respectively) have correspondingly been the subject of pharmaceutical research and development having as its goal the intervention in the processes of leukocyte extravasation to sites of injury and leukocyte movement to distinct targets. For example, it is presently believed that prior to the leukocyte extravasation, which is a mandatory component of the inflammatory response, activation of integrins constitutively expressed on leukocytes occurs and is followed by a tight ligand/receptor interaction between integrins (e.g., LFA-1) and one or several distinct intercellular adhesion molecules (ICAMs) designated ICAM-1, ICAM-2, ICAM-3 or ICAM-4 which are expressed on blood vessel endothelial cell surfaces and on other leukocytes. The interaction of the CAMs with the Leukointegrins is a vital step in the normal functioning of the immune system. Immune processes such as antigen presentation, T-cell mediated cytotoxicity and leukocyte extravasation all require cellular adhesion mediated by ICAMs interacting with the Leukointegrins. See generally Kishimoto, T. K.; Rothlein; R. R. Adv. Pharmacol. 1994, 25, 117-138 and Diamond, M.; Springer, T. Current Biology, 1994, 4, 506-532.

A group of individuals has been identified which lack the appropriate expression of Leukointegrins, a condition termed “Leukocyte Adhesion Deficiency” (Anderson, D.C.; et al, Fed. Proc. 1985, 44, 2671-2677 and Anderson, D. C.; et al., J. Infect. Dis. 1985, 152, 668-689). These individuals are unable to mount a normal inflammatory and/or immune response(s) due to an inability of their cells to adhere to cellular substrates. These data show that immune reactions are mitigated when lymphocytes are unable to adhere in a normal fashion due to the lack of functional adhesion molecules of the CD18 family. By virtue of the fact that LAD patients who lack CD18 cannot mount an inflammatory response, it is believed that antagonism of CD18, CD11/ICAM interactions will also inhibit an inflammatory response.

It has been demonstrated that the antagonism of the interaction between the CAMs and the Leukointegrins can be realized by agents directed against either component. Specifically, blocking of the CAMs, such as for example ICAM-1, or the Leukointegrins, such as for example LFA-1, by antibodies directed against either or both of these molecules effectively inhibits inflammatory responses. In vitro models of inflammation and immune response inhibited by antibodies to CAMs or Leukointegrins include antigen or mitogen-induced lymphocyte proliferation, homotypic aggregation of lymphocytes, T-cell mediated cytolysis and antigen-specific induced tolerance. The relevance of the in vitro studies are supported by in vivo studies with antibodies directed against ICAM-1 or LFA-1. For example, antibodies directed against LFA-1 can prevent thyroid graft rejection and prolong heart allograft survival in mice (Gorski, A.; Immunology Today, 1994, 15, 251-255). Of greater significance, antibodies directed against ICAM-1 have shown efficacy in vivo as anti-inflammatory agents in human diseases such as renal allograft rejection and rheumatoid arthritis (Rothlein, R. R.; Scharschmidt, L., in: Adhesion Molecules; Wegner, C. D., Ed.; 1994, 1-38, Cosimi, C. B.; et al., J. Immunol. 1990, 144, 4604-4612 and Kavanaugh, A.; et al., Arthritis Rheum. 1994, 37, 992-1004) and antibodies directed against LFA-1 have demonstrated immunosuppressive effects in bone marrow transplantation and in the prevention of early rejection of renal allografts (Fischer, A.; et al., Lancet, 1986, 2, 1058-1061 and Le Mauff, B.; et al., Transplantation, 1991, 52, 291-295).

It has also been demonstrated that a recombinant soluble form of ICAM-1 can act as an inhibitor of the ICAM-1 interaction with LFA-1. Soluble ICAM-1 acts as a direct antagonist of CD18,CD11/ICAM-1 interactions on cells and shows inhibitory activity in in vitro models of immune response such as the human mixed lymphocyte response, cytotoxic T cell responses and T cell proliferation from diabetic patients in response to islet cells (Becker, J. C.; et al., J. Immunol. 1993, 151, 7224 and Roep, B. O.; et al., Lancet, 1994, 343, 1590).

Thus, the prior art has demonstrated that large protein molecules which antagonize the binding of the CAMs to the Leukointegrins have therapeutic potential in mitigating inflammatory and immunological responses often associated with the pathogenesis of many autoimmune or inflammatory diseases. However proteins have significant deficiencies as therapeutic agents, including the inability to be delivered orally and potential immunoreactivity which limits the utility of theses molecules for chronic administration. Furthermore, protein-based therapeutics are generally expensive to produce.

Several small molecules have been described in the literature which affect the interaction of CAMs and Leukointegrins. A natural product isolated from the root of Trichilia rubra was found to be inhibitory in an in vitro cell binding assay (Musza, L. L.; et al., Tetrahedron, 1994, 50, 11369-11378). One series of molecules (Boschelli, D. H.; et al., J. Med. Chem. 1994, 37, 717 and Boschelli, D. H.; et al., J. Med. Chem. 1995, 38, 4597-4614) was found to be orally active in a reverse passive Arthus reaction, an induced model of inflammation that is characterized by neutrophil accumulation (Chang, Y. H.; et al., Eur. J. Pharmacol. 1992, 69, 155-164). Another series of molecules was also found to be orally active in a delayed type hypersensitivity reaction in rats (Sanfilippo, P. J.; et al., J. Med. Chem. 1995, 38, 1057-1059). All of these molecules appear to act nonspecifically, either by inhibiting the transcription of ICAM-1 along with other proteins or act intracellularly to inhibit the activation of the Leukointegrins by an unknown mechanism. None of the molecules directly antagonize the interaction of the CAMs with the Leukointegrins. Due to lack of potency, lack of selectivity and lack of a specific mechanism of action, the described small molecules are not likely to be satisfactory for therapeutic use.

It follows that small molecules having the similar ability as large protein molecules to directly and selectively antagonize the binding of the CAMs to the Leukointegrins would make preferable therapeutic agents. WO9839303, WO0130781, U.S. Pat. No. 6,355,664, U.S. 6,350,763 and U.S. 6,353,013 disclose classes of small molecule inhibitors of the interaction of LFA-1 and ICAM-1. WO9911258 discloses that the fungal metabolite mevinolin and derivatives bind to LFA-1 and disrupt the interaction of LFA-1 and ICAM-1. WO9949856 discloses a class of peptidomimetic inhibitors of ICAM binding to LFA-1 and Mac-1.

### BRIEF SUMMARY OF THE INVENTION

A first aspect of the invention comprises a method for treating or preventing inflammatory and immune cell-mediated diseases by the administration of certain novel small molecules. These compounds act by inhibiting the interaction of cellular adhesion molecules, specifically by antagonizing the binding of human intercellular adhesion molecules (including ICAM-1, ICAM-2 and ICAM-3) to the Leukointegrins (especially CD18/CD11a). A second aspect of the invention comprises novel small molecules having the above-noted therapeutic activities. A third aspect of the invention comprises methods for making these novel compounds. A final aspect of the invention comprises pharmaceutical compositions comprising the above-mentioned compounds suitable for the prevention or treatment of inflammatory and immune cell-mediated conditions.

### DETAILED DESCRIPTION OF THE INVENTION

One aspect of the invention comprises compounds of the formula I

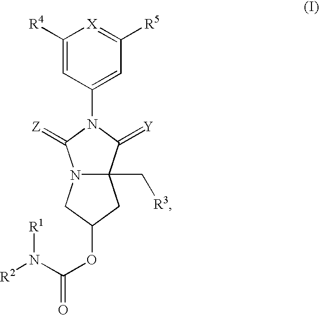

wherein:

Y is an oxygen or sulfur atom;

Z is an oxygen or sulfur atom;

R¹ is selected from the class consisting of:

branched or unbranched alkyl of 1 to 10 carbon atoms, alkenyl of 2 to 10 carbon atoms or cycloalkyl or cycloalkenyl of 3 to 6 carbon atoms, wherein one to three carbon atoms in said alkyl, alkenyl, cycloalkyl or cycloalkenyl may be replaced by —O—, —S—, —S(O)—, —S(O)₂—, —NH— or —N(CH₃)—, and in which said alkyl, alkenyl, cycloalkyl or cycloalkenyl group one to six hydrogen atoms are optionally and independently replaced with:

(i) halogen,

(ii) oxo,

(iii) aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzothiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein one or more hydrogen atoms of said aryl or heteroaryl group are optionally and independently replaced with:

(a) alkyl of 1 to 3 carbon atoms,

(b) —COOH,

(c) a group of the formula —COOR⁶, wherein R⁶ is straight or branched alkyl of 1 to 5 carbon atoms or cycloalkyl of 3 to 5 carbon atoms,

(d) a group of the formula —NR⁷R⁸, wherein R⁷ and R⁸ are each independently a hydrogen atom, alkyl of 1 to 6 carbon atoms, cycloalkyl of 3 to 6 carbon atoms or acyl of 1 to 7 carbon atoms, or wherein R⁷ and R⁸ constitute a saturated hydrocarbon bridge of 3 to 5 carbon atoms which together with the nitrogen atom between them form a heterocyclic ring,

(e) a group of the formula —CONR⁹R¹⁰, wherein R⁹ and R¹⁰ are each independently a hydrogen atom, alkyl of 1 to 6 carbon atoms or cycloalkyl of 3 to 6 carbon atoms, or wherein R⁹ and R¹⁰ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,

(f) a group of the formula —OR¹¹, wherein R¹¹ is a hydrogen atom, or an alkyl or acyl group of 1 to 7 carbon atoms,

(g) a group of the formula —SR¹², wherein R¹² is a hydrogen atom, or an alkyl or acyl group of 1 to 7 carbon atoms,

(h) —CN,

(i) an amidino group of the formula

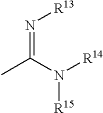

wherein R¹³, R¹⁴ and R¹⁵ are each, independently, a hydrogen atom or alkyl of 1 to 3 carbon atoms and wherein two of R¹³, R¹⁴ and R¹⁵ may additionally constitute a saturated hydrocarbon bridge of 3 to 5 carbon atoms which together with the nitrogen atom(s) between them form a heterocyclic ring,

(j) halogen,

(k) a group of the formula —NHCONHalkyl, wherein the alkyl moiety contains 1 to 3 carbon atoms, or

(l) a group of the formula —NHCOOalkyl, wherein the alkyl moiety contains 1 to 3 carbon atoms,

(iv) a group of the formula —COOR¹⁶, wherein R¹⁶ is hydrogen or straight or branched alkyl of 1 to 7 carbon atoms or cycloalkyl of 3 to 6 carbon atoms,

(v) —CN,

(vi) a group of the formula —CONR¹⁷R¹⁸, wherein R¹⁷ and R¹⁸ are each, independently, a hydrogen atom, alkyl of 1 to 6 carbon atoms or cycloalkyl of 3 to 6 carbon atoms, or wherein R¹⁷ and R¹⁸ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,

(vii) a group of the formula —OR¹⁹, wherein R¹⁹ is a hydrogen atom, or a straight or branched alkyl or acyl group of 1 to 7 carbon atoms, wherein one or more hydrogen atoms of said alkyl or acyl group are optionally replaced with a group independently selected from the class consisting of —OH, —Oalkyl (wherein the alkyl moiety contains 1 to 6 carbon atoms), —NH₂, —NHMe and —NMe₂,

(viii) a group of the formula —SR²⁰, wherein R²⁰ is a hydrogen atom, or an alkyl or acyl group of 1 to 7 carbon atoms, wherein one or more hydrogen atoms of said alkyl or acyl group are optionally replaced with a group independently selected from the class consisting of —OH, —Oalkyl (wherein the alkyl moiety is 1 to 6 carbon atoms), —NH₂, —NHMe and —NMe₂,

(ix) a group of the formula —NR²¹R²², wherein R²¹ and R²² are each, independently,

(a) a hydrogen atom,

(b) straight or branched alkyl or acyl of 1 to 7 carbon atoms or cycloalkyl of 3 to 7 carbon atoms, wherein said one or more hydrogen atoms of said alkyl or acyl group are optionally replaced with a group independently selected from the class consisting of —OH, —Oalkyl (wherein the alkyl moiety is 1 to 6 carbon atoms), —NH₂, —NHMe and —NMe₂,

(c) a group of the formula —(CH₂)ₘCOOH, wherein m is 0, 1 or 2,

(d) a group of the formula —(CH₂)ₙCOOR²³, wherein n is 0, 1 or 2, and wherein R²³ is straight or branched alkyl of 1 to 6 carbon atoms, or

(e) a group of the formula —(CH₂)ₙCONHR²⁴, wherein n is 0, 1 or 2, and wherein R²⁴ is straight or branched alkyl of 1 to 6 carbon atoms,

(x) a saturated, or partially unsaturated heterocyclic group selected from imidazolinyl, imidazolidinyl, pyrrolinyl, pyrrolidinyl, piperidinyl, piperazinyl, morpholinyl, thiomorpholinyl, thiazolidinyl, azepinyl, tetrahydropyranyl, tetrahydrofuranyl, benzodioxolyl, tetrahydrothiophenyl and sulfolanyl, wherein said heterocyclic group is optionally mono- or polysubstituted with:

(a) alkyl of 1 to 3 carbon atoms,

(b) —COOH,

(c) a group of the formula —COOR⁶, wherein R⁶ is straight or branched alkyl of 1 to 5 carbon atoms or cycloalkyl of 3 to 5 carbon atoms,

(d) a group of the formula —C(O)R⁶, wherein R⁶ is straight or branched alkyl of 1 to 5 carbon atoms or cycloalkyl of 3 to 5 carbon atoms,

(e) a group of the formula —NR⁷R⁸, wherein R⁷ and R⁸ are each independently a hydrogen atom, alkyl of 1 to 6 carbon atoms, cycloalkyl of 3 to 6 carbon atoms or acyl of 1 to 7 carbon atoms, or wherein R⁷ and R⁸ constitute a saturated hydrocarbon bridge of 3 to 5 carbon atoms which together with the nitrogen atom between them form a heterocyclic ring,

(f) a group of the formula —CONR⁹R¹⁰, wherein R⁹ and R¹⁰ are each independently a hydrogen atom, alkyl of 1 to 6 carbon atoms or cycloalkyl of 3 to 6 carbon atoms, or wherein R⁹ and R¹⁰ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,

(g) a group of the formula —OR¹¹, wherein R¹¹ is a hydrogen atom, or an alkyl or acyl group of 1 to 7 carbon atoms,

(h) a group of the formula —SR¹², wherein R¹² is a hydrogen atom, or an alkyl or acyl group of 1 to 7 carbon atoms,

(i) —CN,

(j) an amidino group of the formula

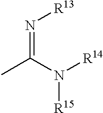

wherein R¹³, R¹⁴ and R¹⁵ are each, independently, a hydrogen atom or alkyl of 1 to 3 carbon atoms and wherein two of R¹³, R¹⁴ and R¹⁵ may additionally constitute a saturated hydrocarbon bridge of 3 to 5 carbon atoms which together with the nitrogen atom(s) between them form a heterocyclic ring,

(k) halogen,

(l) a group of the formula —NHCONHalkyl, wherein the alkyl moiety contains 1 to 3 carbon atoms, or

(m) halogen,

(n) a group of the formula —NHCONHalkyl, wherein the alkyl moiety contains 1 to 3 carbon atoms,

(o) a group of the formula —NHCOOalkyl, wherein the alkyl moiety contains 1 to 3 carbon atoms, or

(p) oxo

or

(xi) a cycloalkyl group of 3 to 7 carbon atoms;

R² is H or C₁₋₃alkyl; or

R¹ and R² together with the nitrogen they are bonded to may form a heterocyclic ring selected from the class consisting of piperidine, pyrrolidine, piperazine, morpholine and thiomorpholine, wherein said heterocyclic ring is optionally mono-, di- or trisubstituted with:

(a) alkyl of 1 to 3 carbon atoms,

(b) —COOH,

(c) a group of the formula —COOR⁶, wherein R⁶ is straight or branched alkyl of 1 to 5 carbon atoms or cycloalkyl of 3 to 5 carbon atoms,

(d) a group of the formula —C(O)R⁶, wherein R⁶ is straight or branched alkyl of 1 to 5 carbon atoms or cycloalkyl of 3 to 5 carbon atoms,

(e) a group of the formula —NR⁷R⁸, wherein R⁷ and R⁸ are each independently a hydrogen atom, alkyl of 1 to 6 carbon atoms, cycloalkyl of 3 to 6 carbon atoms or acyl of 1 to 7 carbon atoms, or wherein R⁷ and R⁸ constitute a saturated hydrocarbon bridge of 3 to 5 carbon atoms which together with the nitrogen atom between them form a heterocyclic ring,

(f) a group of the formula —CONR⁹R¹⁰, wherein R⁹ and R¹⁰ are each independently a hydrogen atom, alkyl of 1 to 6 carbon atoms or cycloalkyl of 3 to 6 carbon atoms, or wherein R⁹ and R¹⁰ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,

(g) a group of the formula —OR¹¹, wherein R¹¹ is a hydrogen atom, or an alkyl or acyl group of 1 to 7 carbon atoms,

(h) a group of the formula —SR¹², wherein R¹² is a hydrogen atom, or an alkyl or acyl group of 1 to 7 carbon atoms,

(i) —CN,

(j) an amidino group of the formula

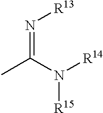

wherein R¹³, R¹⁴ and R¹⁵ are each, independently, a hydrogen atom or alkyl of 1 to 3 carbon atoms and wherein two of R¹³, R¹⁴ and R¹⁵ may additionally constitute a saturated hydrocarbon bridge of 3 to 5 carbon atoms which together with the nitrogen atom(s) between them form a heterocyclic ring,

(k) halogen,

(l) a group of the formula —NHCONHalkyl, wherein the alkyl moiety contains 1 to 3 carbon atoms, or

(m) a group of the formula —NHCONHalkyl, wherein the alkyl moiety contains 1 to 3 carbon atoms,

(n) a group of the formula —NHCOOalkyl, wherein the alkyl moiety contains 1 to 3 carbon atoms, or

(o) oxo

R³ is

aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzothiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein one or more of the hydrogen atoms of said aryl or heteroaryl group is optionally and independently replaced with:

(A) R⁴⁴, which is aryl or heteroaryl selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzothiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein one or more of the hydrogen atoms of said aryl or heteroaryl group is optionally and independently replaced with:

(i) branched or unbranched alkyl of 1 to 6 carbon atoms or cycloalkyl of 3 to 6 carbon atoms, which alkyl or cycloakyl group is optionally mono- or polysubstituted with halogen or oxo,

(ii) a group of the formula COOR⁴⁵, wherein R⁴⁵ is straight or branched alkyl of 1 to 5 carbon atoms or cycloalkyl of 3 to 5 carbon atoms,

(iii) a group of the formula —NR⁴⁶R⁴⁷, wherein R⁴⁶ and R⁴⁷ are each, independently, a hydrogen atom, alkyl or fluoroalkyl of 1 to 6 carbon atoms, cycloalkyl of 3 to 6 carbon atoms or acyl of 1 to 7 carbon atoms, or wherein R⁴⁶ and R⁴⁷ constitute a saturated hydrocarbon bridge of 3 to 5 carbon atoms which together with the nitrogen atom between them form a heterocyclic ring,

(iv) a group of the formula —CONR⁴⁸R⁴⁹, wherein R⁴⁸ and R⁴⁹ are each, independently, a hydrogen atom, alkyl or fluoroalkyl of 1 to 6 carbon atoms or cycloalkyl of 3 to 6 carbon atoms, or wherein R⁴⁸ and R⁴⁹ constitute a saturated hydrocarbon bridge of 3 to 5 carbon atoms which together with the nitrogen atom between them form a heterocyclic ring,

(v) a group of the formula —OR⁵⁰, wherein R⁵⁰ is a hydrogen atom, or an alkyl, fluoroalkyl or acyl group of 1 to 7 carbon atoms,

(vi) a group of the formula —SR⁵¹, wherein R⁵¹ is a hydrogen atom, or an alkyl, fluoroalkyl or acyl group of 1 to 7 carbon atoms,

(vii) —CN,

(viii) nitro, or

(ix) halogen,

(B) methyl, which is optionally mono- or polysubstituted with fluorine atoms and additionally is optionally monosubstituted with R⁴⁴,

(C) branched or unbranched alkyl of 2 to 6 carbon atoms or cycloalkyl of 3 to 6 carbon atoms, which alkyl or cycloakyl group is optionally mono- or polysubstituted with halogen or oxo,

(D) a group of the formula —COOR⁵², wherein R⁵² is straight or branched alkyl of 1 to 5 carbon atoms or cycloalkyl of 3 to 5 carbon atoms,

(E) a group of the formula —NR⁵³R⁵⁴, wherein R⁵³ and R⁵⁴ are each, independently, a hydrogen atom, alkyl or fluoroalkyl of 1 to 6 carbon atoms, cycloalkyl of 3 to 6 carbon atoms or acyl of 1 to 7 carbon atoms, or wherein R⁵³ and R⁵⁴ constitute a saturated hydrocarbon bridge of 3 to 5 carbon atoms which together with the nitrogen atom between them form a heterocyclic ring, and wherein one of R⁵³ and R⁵⁴ may additionally be the group R⁴⁴,

(F) a group of the formula —CONR⁵⁵R⁵⁶, wherein R⁵⁵ and R⁵⁶ are each, independently, a hydrogen atom, alkyl or fluoroalkyl of 1 to 6 carbon atoms or cycloalkyl of 3 to 6 carbon atoms, or wherein R⁵⁵ and R⁵⁶ constitute a saturated hydrocarbon bridge of 3 to 5 carbon atoms which together with the nitrogen atom between them form a heterocyclic ring, and wherein one of R⁵⁵ and R⁵⁶ may additionally be the group R⁴⁴,

(G) a group of the formula —COR⁵⁷, wherein R⁵⁷ is a hydrogen atom, straight or branched alkyl of 1 to 5 carbon atoms, cycloalkyl of 3 to 5 carbon atoms or R⁴⁴,

(H) a group of the formula —OR⁵⁸, wherein R⁵⁸ is a hydrogen atom, an alkyl, fluoroalkyl or acyl group of 1 to 7 carbon atoms, or R⁴⁴,

(I) a group of the formula —SR⁵⁹, wherein R⁵⁹ is a hydrogen atom, an alkyl, fluoroalkyl or acyl group of 1 to 7 carbon atoms, or R⁴⁴,

(J) —CN,

(K) nitro, or

(L) halogen;

R⁴ is Cl or trifluoromethyl;

X is ═N— or ═C(R⁶⁰)— wherein R⁶⁰ is a hydrogen, fluorine, chlorine, bromine or iodine atom, methyl or trifluoromethyl; and,

R⁵ is a hydrogen, fluorine, chlorine, bromine or iodine atom, methyl, —CN, nitro or trifluoromethyl, with the condition that when X is ═N— or ═C(H)—, R⁵ is chlorine, trifluoromethyl, —CN or nitro;

and pharmaceutically acceptable salts thereof.

Preferred are compounds of the formula I

wherein:

Y is an oxygen atom;

Z is an oxygen atom;

R¹ is selected from the class consisting of:

branched or unbranched alkyl of 1 to 10 carbon atoms or alkenyl of 2 to 10 carbon atoms, wherein one to three carbon atoms in said alkyl, alkenyl, cycloalkyl or cycloalkenyl may be replaced by —O—, —NH— or —N(CH₃)—, and in which said alkyl or alkenyl group one to six hydrogen atoms are optionally and independently replaced with:

(i) halogen,

(ii) oxo,

(iii) aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, pyrazinyl, isoindolyl, benzo[b]furanyl, benzo[b]thiophenyl, benzothiazolyl, benzimidazolyl, quinolinyl isoquinolinyl and quinazolinyl, wherein one or more hydrogen atoms of said aryl or heteroaryl group are optionally and independently replaced with:

(a) alkyl of 1 to 3 carbon atoms,

(b) —COOH,

(c) a group of the formula —COOR⁶, wherein R⁶ is straight or branched alkyl of 1 to 5 carbon atoms or cycloalkyl of 3 to 5 carbon atoms,

(d) a group of the formula —NR⁷R⁸, wherein R⁷ and R⁸ are each independently a hydrogen atom, alkyl of 1 to 6 carbon atoms, cycloalkyl of 3 to 6 carbon atoms or acyl of 1 to 7 carbon atoms, or wherein R⁷ and R⁸ constitute a saturated hydrocarbon bridge of 3 to 5 carbon atoms which together with the nitrogen atom between them form a heterocyclic ring,

(e) a group of the formula —CONR⁹R¹⁰, wherein R⁹ and R¹⁰ are each independently a hydrogen atom, alkyl of 1 to 6 carbon atoms or cycloalkyl of 3 to 6 carbon atoms, or wherein R⁹ and R¹⁰ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,

(f) a group of the formula —OR¹¹, wherein R¹¹ is a hydrogen atom, or an alkyl or acyl group of 1 to 7 carbon atoms,

(g) a group of the formula —SR¹², wherein R¹² is a hydrogen atom, or an alkyl or acyl group of 1 to 7 carbon atoms,

(h) —CN,

(i) halogen,

(k) a group of the formula —NHCONHalkyl, wherein the alkyl moiety contains 1 to 3 carbon atoms, or

(l) a group of the formula —NHCOOalkyl, wherein the alkyl moiety contains 1 to 3 carbon atoms,

(iv) —CN,

(v) a group of the formula —OR¹⁹, wherein R¹⁹ is a hydrogen atom, or a straight or branched alkyl or acyl group of 1 to 7 carbon atoms, wherein one or more hydrogen atoms of said alkyl or acyl group are optionally replaced with a group independently selected from the class consisting of —OH, —Oalkyl (wherein the alkyl moiety contains 1 to 6 carbon atoms), —NH₂, —NHMe and —NMe₂,

(vi) a group of the formula —SR²⁰, wherein R²⁰ is a hydrogen atom, or an alkyl or acyl group of 1 to 7 carbon atoms, wherein one or more hydrogen atoms of said alkyl or acyl group are optionally replaced with a group independently selected from the class consisting of —OH, —Oalkyl (wherein the alkyl moiety is 1 to 6 carbon atoms), —NH₂, —NHMe and —NMe₂,

(vii) a group of the formula —NR²¹R²², wherein R²¹ and R²² are each, independently,

(a) a hydrogen atom,

(b) straight or branched alkyl or acyl of 1 to 7 carbon atoms or cycloalkyl of 3 to 7 carbon atoms, wherein said one or more hydrogen atoms of said alkyl or acyl group are optionally replaced with a group independently selected from the class consisting of —OH, —Oalkyl (wherein the alkyl moiety is 1 to 6 carbon atoms), —NH₂, —NHMe and —NMe₂,

(x) a saturated, or partially unsaturated heterocyclic group selected from imidazolinyl, imidazolidinyl, pyrrolinyl, pyrrolidinyl, piperidinyl, piperazinyl, morpholinyl, thiomorpholinyl, thiazolidinyl, azepinyl, tetrahydropyranyl, tetrahydrofuranyl, benzodioxolyl, tetrahydrothiophenyl and sulfolanyl, wherein said heterocyclic group is optionally mono- or polysubstituted with:

(a) alkyl of 1 to 3 carbon atoms,

(b) —COOH,

(e) a group of the formula —COOR⁶, wherein R⁶ is straight or branched alkyl of 1 to 5 carbon atoms or cycloalkyl of 3 to 5 carbon atoms,

(f) a group of the formula —C(O)R⁶, wherein R⁶ is straight or branched alkyl of 1 to 5 carbon atoms or cycloalkyl of 3 to 5 carbon atoms,

(e) a group of the formula —NR⁷R⁸, wherein R⁷ and R⁸ are each independently a hydrogen atom, alkyl of 1 to 6 carbon atoms, cycloalkyl of 3 to 6 carbon atoms or acyl of 1 to 7 carbon atoms, or wherein R⁷ and R⁸ constitute a saturated hydrocarbon bridge of 3 to 5 carbon atoms which together with the nitrogen atom between them form a heterocyclic ring,

(f) a group of the formula —CONR⁹R¹⁰, wherein R⁹ and R¹⁰ are each independently a hydrogen atom, alkyl of 1 to 6 carbon atoms or cycloalkyl of 3 to 6 carbon atoms, or wherein R⁹ and R¹⁰ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,

(g) a group of the formula —OR¹¹, wherein R¹¹ is a hydrogen atom, or an alkyl or acyl group of 1 to 7 carbon atoms,

(h) a group of the formula —SR¹², wherein R¹² is a hydrogen atom, or an alkyl or acyl group of 1 to 7 carbon atoms,

(i) —CN,

(j) halogen,

(k) a group of the formula —NHCONHalkyl, wherein the alkyl moiety contains 1 to 3 carbon atoms, or

(l) halogen,

(m) a group of the formula —NHCONHalkyl, wherein the alkyl moiety contains 1 to 3 carbon atoms,

(n) a group of the formula —NHCOOalkyl, wherein the alkyl moiety contains 1 to 3 carbon atoms, or

(o) oxo or

(di) a cycloalkyl group of 3 to 7 carbon atoms;

R² is H; or

R¹ and R² together with the nitrogen they are bonded to may form a heterocyclic ring selected from the class consisting of piperidine, pyrrolidine, piperazine, morpholine and thiomorpholine, wherein said heterocyclic ring is optionally mono-, di- or trisubstituted with:

(a) alkyl of 1 to 3 carbon atoms,

(b) —COOH,

(c) a group of the formula —COOR⁶, wherein R⁶ is straight or branched alkyl of 1 to 5 carbon atoms or cycloalkyl of 3 to 5 carbon atoms,

(d) a group of the formula —C(O)R⁶, wherein R⁶ is straight or branched alkyl of 1 to 5 carbon atoms or cycloalkyl of 3 to 5 carbon atoms,

(e) a group of the formula —NR⁷R⁸, wherein R⁷ and R⁸ are each independently a hydrogen atom, alkyl of 1 to 6 carbon atoms, cycloalkyl of 3 to 6 carbon atoms or acyl of 1 to 7 carbon atoms,

(f) a group of the formula —CONR⁹R¹⁰, wherein R⁹ and R¹⁰ are each independently a hydrogen atom, alkyl of 1 to 6 carbon atoms or cycloalkyl of 3 to 6 carbon atoms

(g) a group of the formula —OR¹¹, wherein R¹¹ is a hydrogen atom, or an alkyl or acyl group of 1 to 7 carbon atoms,

(h) —CN,

(i) halogen,

R³ is

aryl or heteroaryl which is selected from the class consisting of phenyl, pyridyl and pyrimidinyl, wherein one or more of the hydrogen atoms of said aryl or heteroaryl group is optionally and independently replaced with:

(A) R⁴⁴, which is aryl or heteroaryl selected from the class consisting of phenyl, thiophenyl, pyridyl, pyrimidinyl, furyl, pyrrolyl, oxazolyl and thiazolyl, wherein one or more of the hydrogen atoms of said aryl or heteroaryl group is optionally and independently replaced with:

(i) branched or unbranched alkyl of 1 to 6 carbon atoms or cycloalkyl of 3 to 6 carbon atoms, which alkyl or cycloakyl group is optionally mono- or polysubstituted with halogen or oxo,

(ii) —CN,

(iii) nitro, or

(iv) halogen,

(B) methyl, which is optionally mono- or polysubstituted with fluorine atoms and additionally is optionally monosubstituted with R⁴⁴,

(C) branched or unbranched alkyl of 2 to 6 carbon atoms or cycloalkyl of 3 to 6 carbon atoms, which alkyl or cycloakyl group is optionally mono- or polysubstituted with halogen or oxo,

(D) a group of the formula —COOR⁵², wherein R⁵² is straight or branched alkyl of 1 to 5 carbon atoms or cycloalkyl of 3 to 5 carbon atoms,

(E) a group of the formula —COR⁵⁷, wherein R⁵⁷ is a hydrogen atom, straight or branched alkyl of 1 to 5 carbon atoms, cycloalkyl of 3 to 5 carbon atoms or R⁴⁴,

(F) a group of the formula —OR⁵⁸, wherein R⁵⁸ is a hydrogen atom, an alkyl, fluoroalkyl or acyl group of 1 to 7 carbon atoms, or R⁴⁴,

(G) —CN,

(H) nitro, or

(I) halogen;

R⁴ is Cl or trifluoromethyl;

X is ═N— or ═C(R⁶⁰)— wherein R⁶⁰ is a hydrogen, fluorine, chlorine, bromine or iodine atom, methyl or trifluoromethyl; and,

R⁵ is a fluorine, chlorine, bromine or iodine atom, methyl, —CN, nitro or trifluoromethyl, with the condition that when X is ═N— or ═C(H)—, R⁵ is chlorine, trifluoromethyl, —CN or nitro;

and pharmaceutically acceptable salts thereof.

More preferred are compounds of the formula I

wherein:

Y is an oxygen atom;

Z is an oxygen atom;

R¹ is selected from the class consisting of:

branched or unbranched alkyl of 1 to 10 carbon atoms or alkenyl of 2 to 10 carbon atoms, wherein one to three carbon atoms in said alkyl, alkenyl, cycloalkyl or cycloalkenyl may be replaced by —O—, —NH— or —N(CH₃)—, and in which said alkyl or alkenyl group one to six hydrogen atoms are optionally and independently replaced with:

(i) halogen,

(ii) oxo,

(iv) aryl or heteroaryl which is selected from the class consisting of phenyl, indolyl, pyridyl, pyrimidinyl, pyrrolyl, oxazolyl, thiazolyl, pyrazolyl, imidazolyl, pyrazinyl, benzothiazolyl, benzimidazolyl, quinolinyl isoquinolinyl and quinazolinyl, wherein one or more hydrogen atoms of said aryl or heteroaryl group are optionally and independently replaced with:

(a) alkyl of 1 to 3 carbon atoms,

(b) —COOH,

(c) a group of the formula —NR⁷R⁸, wherein R⁷ and R⁸ are each independently a hydrogen atom or a methyl group,

(d) a group of the formula —CONR⁹R¹⁰, wherein R⁹ and R¹⁰ are each independently a hydrogen atom or a methyl group,

(e) a group of the formula —OR¹¹, wherein R¹¹ is a hydrogen atom, or an alkyl or acyl group of 1 to 7 carbon atoms,

(f) —CN,

(i) halogen,

(iv) —CN,

(v) a group of the formula —OR¹⁹, wherein R¹⁹ is a hydrogen atom, or a straight or branched alkyl or acyl group of 1 to 7 carbon atoms, wherein one or more hydrogen atoms of said alkyl or acyl group are optionally replaced with a group independently selected from the class consisting of —OH, —Oalkyl (wherein the alkyl moiety contains 1 to 6 carbon atoms), —NH₂, —NHMe and —NMe₂,

(vi) a group of the formula —NR²¹R²², wherein R²¹ and R²² are each, independently,

(a) a hydrogen atom,

(b) methyl

(x) a saturated, or partially unsaturated heterocyclic group selected from imidazolinyl, pyrrolidinyl, piperidinyl, piperazinyl, morpholinyl, thiomorpholinyl, tetrahydropyranyl and tetrahydrofuranyl, wherein said heterocyclic group is optionally mono- or polysubstituted with:

(a) alkyl of 1 to 3 carbon atoms,

(b) —COOH,

(c) a group of the formula —C(O)R⁶, wherein R⁶ is straight or branched alkyl of 1 to 5 carbon atoms or cycloalkyl of 3 to 5 carbon atoms,

(d) a group of the formula —NR⁷R⁸, wherein R⁷ and R⁸ are each independently a hydrogen atom or a methyl group,

(e) a group of the formula —CONR⁹R¹⁰, wherein R⁹ and R¹⁰ are each independently a hydrogen atom or a methyl group,

(f) a group of the formula —OR¹¹, wherein R¹¹ is a hydrogen atom, or an alkyl or acyl group of 1 to 7 carbon atoms,

(g) —CN, or

(h) halogen,

R² is H; or

R¹ and R² together with the nitrogen they are bonded to may form a heterocyclic ring selected from the class consisting of piperidine, pyrrolidine, piperazine and morpholine, wherein said heterocyclic ring is optionally mono-, di- or trisubstituted with:

(a) alkyl of 1 to 3 carbon atoms,

(b) —COOH,

(c) a group of the formula —C(O)R⁶, wherein R⁶ is straight or branched alkyl of 1 to 5 carbon atoms or cycloalkyl of 3 to 5 carbon atoms,

(d) a group of the formula —NR⁷R⁸, wherein R⁷ and R⁸ are each independently a hydrogen atom or a methyl group

(e) a group of the formula —CONR⁹R¹⁰, wherein R⁹ and R¹⁰ are each independently a hydrogen atom or a methyl group,

(f) a group of the formula —OR¹¹, wherein R¹¹ is a hydrogen atom, or an alkyl or acyl group of 1 to 7 carbon atoms,

(h) —CN,

(i) halogen,

R³ is

phenyl, which is optionally substituted in the 4-position with:

(A) R⁴⁴, which is aryl or heteroaryl selected from the class consisting of phenyl, thiophenyl, pyridyl, pyrimidinyl and furyl, wherein one or more of the hydrogen atoms of said aryl or heteroaryl group is optionally and independently replaced with:

(i) branched or unbranched alkyl of 1 to 6 carbon atoms or cycloalkyl of 3 to 6 carbon atoms, which alkyl or cycloakyl group is optionally mono- or polysubstituted with halogen or oxo,

(ii) —CN,

(iii) nitro, or

(iv) halogen,

(B) methyl,

(C) branched or unbranched alkyl of 2 to 6 carbon atoms or cycloalkyl of 3 to 6 carbon atoms, which alkyl or cycloakyl group is optionally monosubstituted with halogen or oxo,

(D) a group of the formula —COOR⁵², wherein R⁵² is straight or branched alkyl of 1 to 5 carbon atoms or cycloalkyl of 3 to 5 carbon atoms,

(E) a group of the formula —COR⁵⁷, wherein R⁵⁷ is a hydrogen atom, straight or branched alkyl of 1 to 5 carbon atoms, cycloalkyl of 3 to 5 carbon atoms,

(F) a group of the formula —OR⁵⁸, wherein R⁵⁸ is a hydrogen atom, an alkyl, fluoroalkyl or acyl group of 1 to 7 carbon atoms,

(G) —CN,

(H) nitro, or

(I) halogen;

R³ is Cl;

X is ═C(H)—; and,

R⁴ is Cl;

and pharmaceutically acceptable salts thereof.

Even more preferred are compounds of the formula I

wherein:

Y is an oxygen atom;

Z is an oxygen atom;

R¹ is selected from the class consisting of:

branched or unbranched alkyl of 1 to 10 carbon atoms or alkenyl of 2 to 10 carbon atoms, wherein one to three carbon atoms in said alkyl, alkenyl, cycloalkyl or cycloalkenyl may be replaced by —O—, —NH— or N(CH₃)—, and in which said alkyl or alkenyl group one to six hydrogen atoms are optionally and independently replaced with:

(i) halogen,

(ii) oxo,

(v) aryl or heteroaryl which is selected from the class consisting of phenyl, indolyl, pyridyl, pyrimidinyl, oxazolyl, thiazolyl, pyrazolyl, imidazolyl, pyrazinyl, benzothiazolyl and benzimidazolyl, wherein one or more hydrogen atoms of said aryl or heteroaryl group are optionally and independently replaced with:

(a) alkyl of 1 to 3 carbon atoms,

(b) —COOH,

(c) a group of the formula —NR⁷R⁸, wherein R⁷ and R⁸ are each independently a hydrogen atom or a methyl group,

(d) a group of the formula —OR¹¹, wherein R¹¹ is a hydrogen atom or a methyl group,

(e) —CN,

(i) halogen,

(iv) —CN,

(v) a group of the formula —OR¹⁹, wherein R¹⁹ is a hydrogen atom, a methyl group or an acetyl group,

(vi) a group of the formula —NR²¹R²², wherein R²¹ and R²² are each, independently,

(a) a hydrogen atom,

(b) methyl

(vii) a saturated, or partially unsaturated heterocyclic group selected from piperidinyl, piperazinyl and morpholinyl, wherein said heterocyclic group is optionally mono- or polysubstituted with:

(a) alkyl of 1 to 3 carbon atoms,

(b) —COOH,

(c) a group of the formula —C(O)R⁶, wherein R⁶ is straight or branched alkyl of 1 to 5 carbon atoms or cycloalkyl of 3 to 5 carbon atoms,

(d) a group of the formula —OR¹¹, wherein R¹¹ is a hydrogen atom, or an alkyl or acyl group of 1 to 7 carbon atoms, or

(e) halogen,

R² is H; or

R¹ and R² together with the nitrogen they are bonded to may form a heterocyclic ring selected from the class consisting of piperidine, pyrrolidine, piperazine and morpholine, wherein said heterocyclic ring is optionally mono-, di- or trisubstituted with:

(a) alkyl of 1 to 3 carbon atoms,

(b) —COOH,

(c) a group of the formula —C(O)R⁶, wherein R⁶ is straight or branched alkyl of 1 to 5 carbon atoms or cycloalkyl of 3 to 5 carbon atoms,

(d) a group of the formula —NR⁷R⁸, wherein R⁷ and R⁸ are each independently a hydrogen atom or a methyl group, or

(e) a group of the formula —OR¹¹, wherein R¹¹ is a hydrogen atom, or an alkyl or acyl group of 1 to 7 carbon atoms,

R³ is

phenyl, which is optionally substituted in the 4-position with:

(A) R⁴⁴, which is aryl or heteroaryl selected from the class consisting of phenyl, pyridyl and pyrimidinyl,

(B) methyl,

(C) CN

(D) nitro, or

(E) halogen;

R³ is Cl;

X is ═C(H); and,

R⁴ is Cl;

and pharmaceutically acceptable salts thereof.

Further preferred are compounds of the formula I

wherein:

Y is an oxygen atom;

Z is an oxygen atom;

R¹ is selected from the class consisting of:

branched or unbranched alkyl of 1 to 8 carbon atoms or alkenyl of 2 to 8 carbon atoms, wherein one to three carbon atoms in said alkyl, alkenyl, cycloalkyl or cycloalkenyl may be replaced by —O—, and in which said alkyl or alkenyl group one to six hydrogen atoms are optionally and independently replaced with:

(i) oxo,

(ii) aryl or heteroaryl which is selected from the class consisting of phenyl, pyridyl and imidazolyl, wherein one or more hydrogen atoms of said aryl or heteroaryl group are optionally and independently replaced with:

(a) methyl,

(b) —COOH,

(c) a group of the formula OR¹¹, wherein R¹¹ is a hydrogen atom or a methyl group,

(iii) a group of the formula —OR¹⁹, wherein R¹⁹ is a hydrogen atom, a methyl group or an acetyl group,

R² is H; or

R¹ and R² together with the nitrogen they are bonded to may form a heterocyclic ring selected from the class consisting of piperidine, pyrrolidine, piperazine and morpholine, wherein said heterocyclic ring is optionally mono-, di- or trisubstituted with:

(a) alkyl of 1 to 3 carbon atoms,

(b) —COOH,

(c) a group of the formula —C(O)R⁶, wherein R⁶ is straight or branched alkyl of 1 to 5 carbon atoms or cycloalkyl of 3 to 5 carbon atoms,

(d) a group of the formula —OR¹¹, wherein R¹¹ is a hydrogen atom, a methyl group or an acetyl group,

R³ is

phenyl, which is optionally substituted in the 4-position with:

(A) R⁴⁴, which is aryl or heteroaryl selected from the class consisting of phenyl, pyridyl and pyrimidinyl,

(B) methyl,

(C) CN

(D) nitro, or

(E) halogen;

R⁴ is Cl;

X is ═C(H); and,

R⁵ is Cl;

and pharmaceutically acceptable salts thereof.

Especially preferred compounds include:

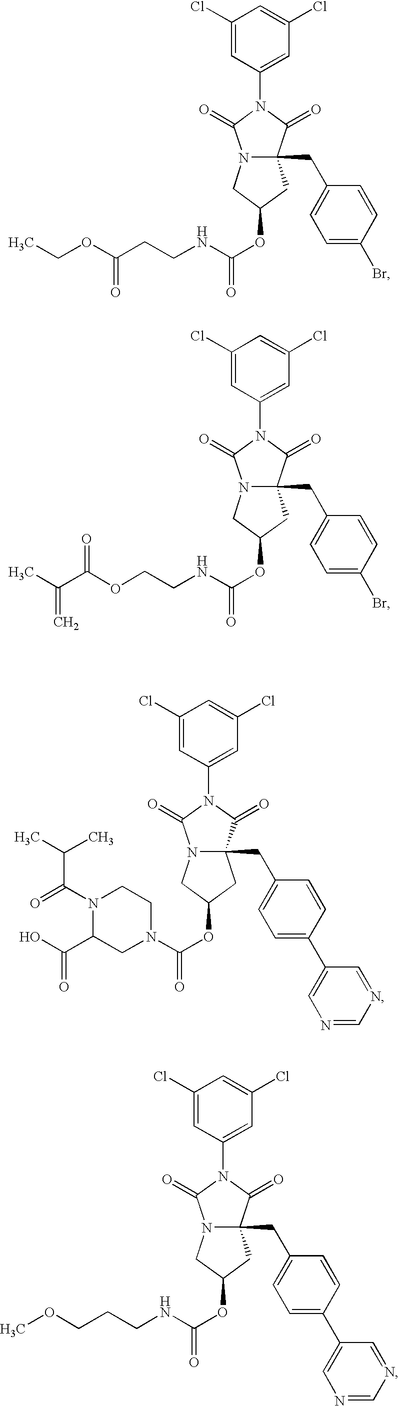

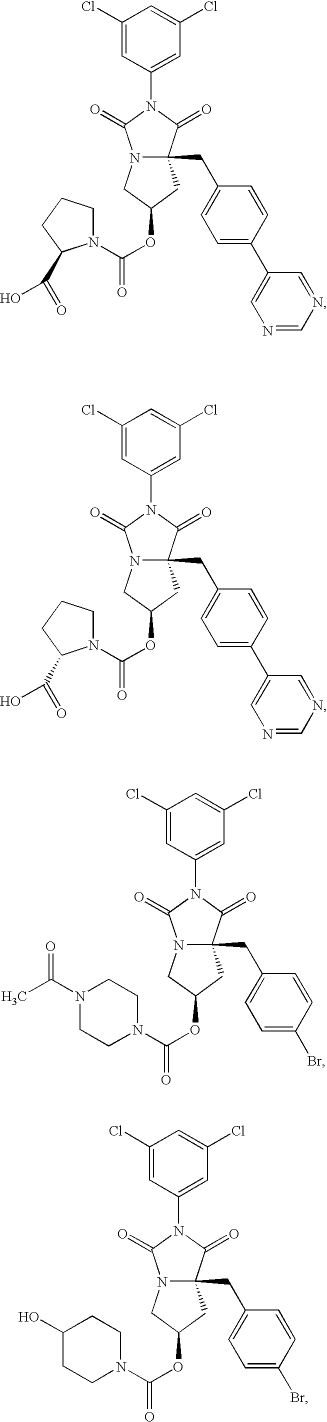

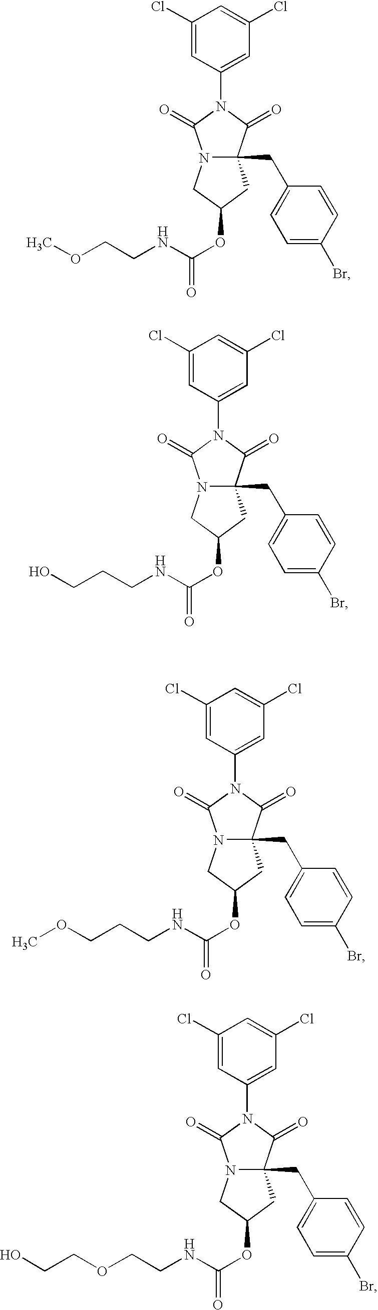

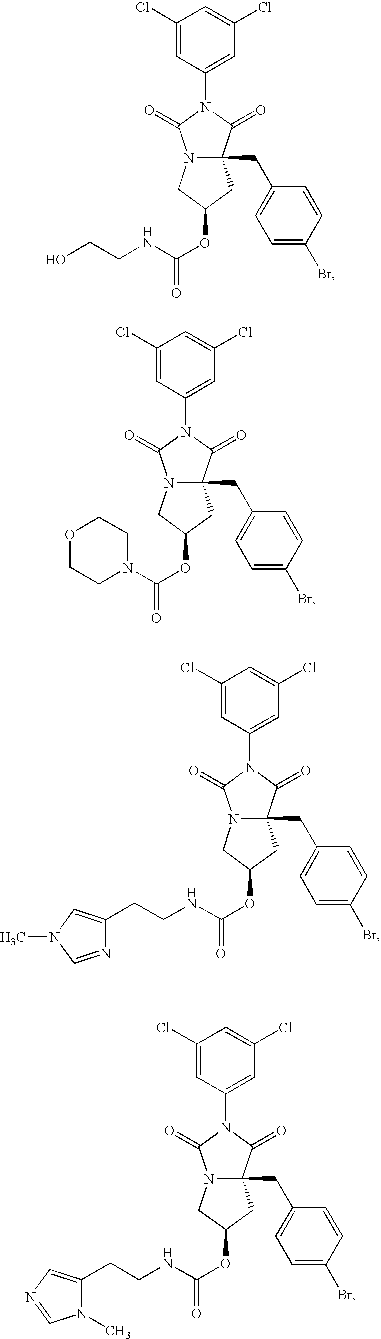

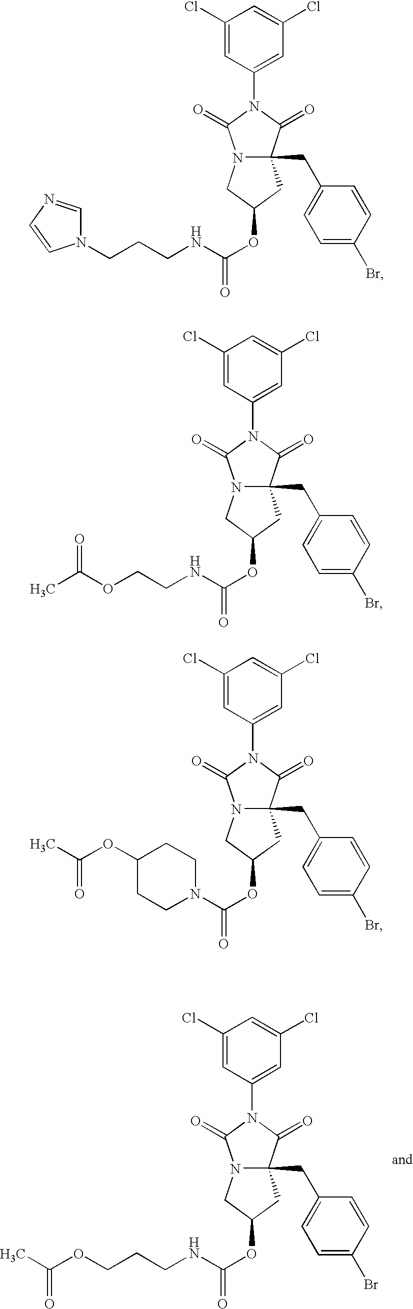

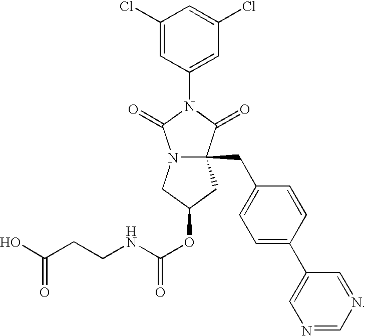

It will be appreciated that the compounds of formula I have at least two chiral centers. Ultimately preferred are those compounds of formula I with the absolute stereochemistry depicted below in formula Ia.

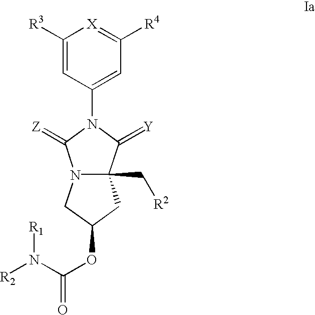

General Synthetic Methods

Compounds of the invention may be prepared by the general methods described below. Typically, reaction progress may be monitored by thin layer chromatography (TLC) if desired. If desired, intermediates and products may be purified by chromatography on silica gel and/or recrystallization, and characterized by one or more of the following techniques: NMR, mass spectroscopy and melting point. Starting materials and reagents are either commercially available or may be prepared by one skilled in the art using methods described in the chemical literature.

All the compounds of the invention may be derived from a common intermediate, A (Scheme I). This may be synthesized from the commercially available N-benzyloxycarbonyl-4-hydroxy-L-proline by a published method (Weber et al., Helv. Chim. Acta, 1985, 68, 155-161).

R₂CH₂— is introduced by alkylation of intermediate A with a suitable alkylating agent bearing R₂CH₂—, such as R₂CH₂Br or R₂CH₂I, in the presence of a suitable base such as lithium diisopropyl amide (LDA) to provide B. This is followed by hydrolysis of the N, O acetal by treatment with an acid, such as 6N HCl, as illustrated in Scheme I giving the hydroxyproline salt C.

The bicyclic framework is completed by reacting amino acid C with an appropriately substituted phenylisocyanate (D) in the presence of a base such as sodium hydroxide, providing intermediate E, followed by intramolecular condensation with a coupling reagent such as EDC to provide F. Intermediate F is used to prepare compounds of formula (I) by the methods described below.

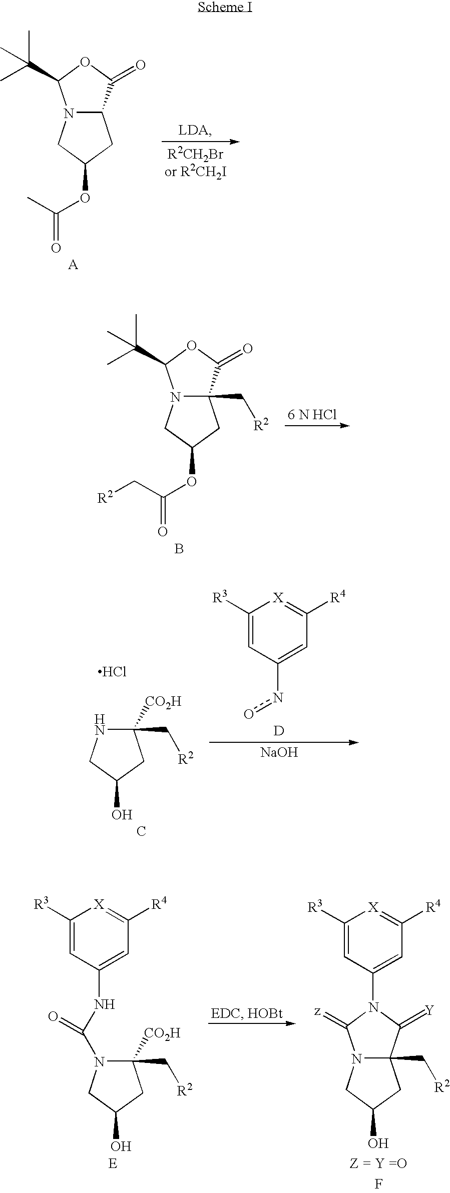

If one desires to invert the stereochemistry of the hydroxyl group in F, one may use Mitsunobu conditions, reacting F with triphenyl phosphine, diethyl azodicarboxylate (DEAD) and a carboxylic acid to provide the ester with inverted stereochemistry G as illustrated in Scheme II, Method A. Hydrolysis under standard conditions such as treating G with aqueous base provides the desired F′.

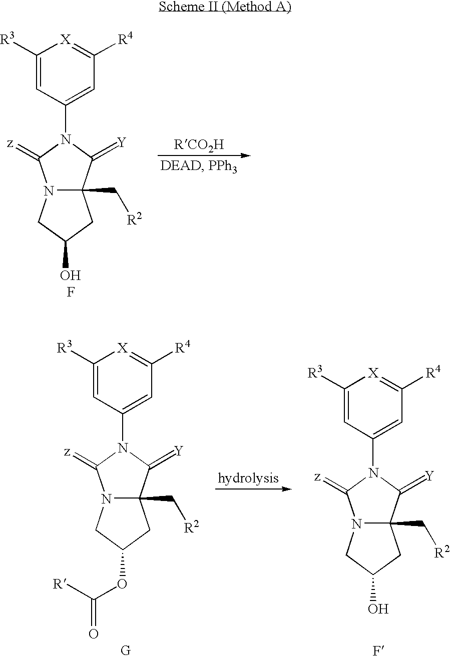

Compounds of formula (I) may then be prepared by methods known in the art for preparing carbamates. For example, reaction of F or F′ with p-nitrophenyl chloroformate, in the presence of a suitable base such as triethylamine, followed by reaction with the desired amine provides the desired compound of formula (I) as illustrated in Scheme III, Method B.

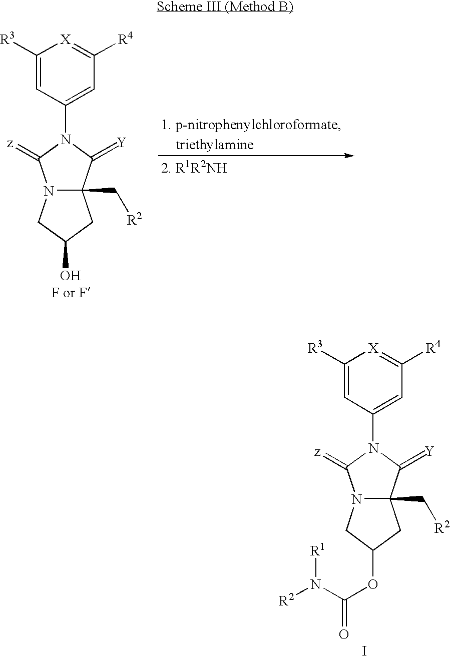

### SYNTHETIC EXAMPLES

### Example 1

7a-(4-Bromo-benzyl-2-(3,5-dichloro-phenyl)-6-hydroxy-tetrahydro-pyrrolo[1,2-c]imidazole-1,3-dione

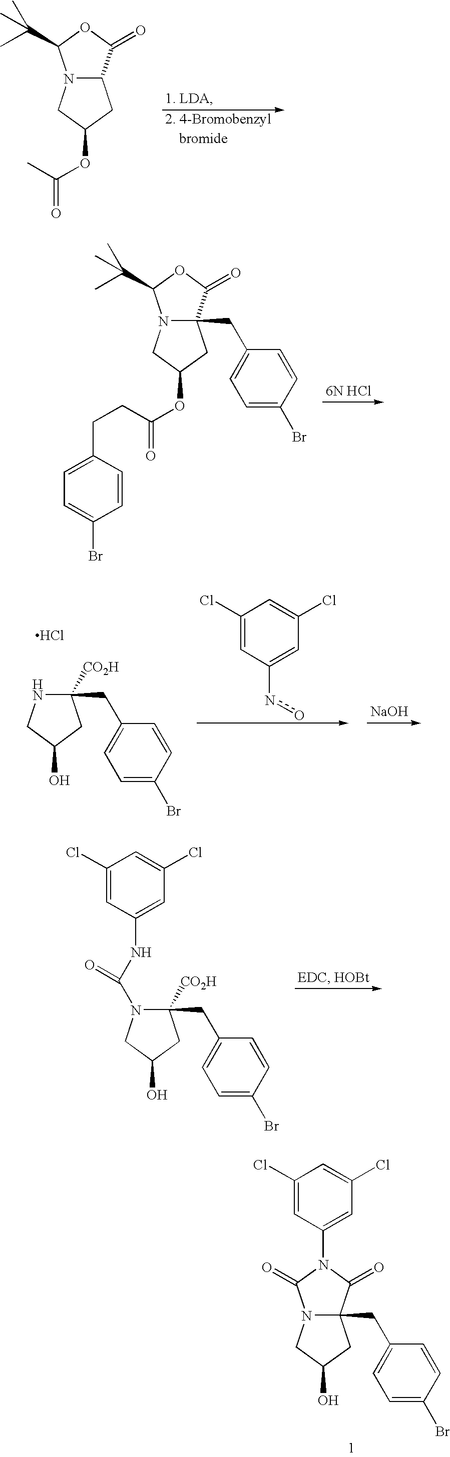

Acetic acid 3-tert-butyl-1-oxo-tetrahydro-pyrrolo[1,2-c]oxazol-6-yl ester (Weber et al., Helv. Chim. Acta, 1985, 68, 155) (4.5 g, 18.6 mmol) was dissolved in THF (200 mL) and the solution was cooled to −78° C. To this solution was added dropwise a 2 N solution of LDA in THF/heptane/ethylbenzene (19.5 mL, 39 mmol). The mixture was stirred for 1 h at this temperature after which 4-bromobenzyl bromide (13.95 g, 55.8 mmol) dissolved in THF (20 mL) was added carefully. The solution was kept at −78° C. for another hour after which it was warmed up to −20° C. The mixture was diluted with ethyl ether (125 mL) and saturated aqueous ammonium chloride solution (50 mL) and the organic phase was extracted three times with ethyl ether. The combined organic phases were washed with brine and dried over MgSO₄. The volatiles were evaporated under reduced pressure and the residue was extracted with hexanes three times to yield the crude bis-(4-bromobenzyl)intermediate.

This was dissolved in 6N HCl (40 mL) and the mixture was refluxed for 90 min. The aqueous phase was extracted three times with methylene chloride and evaporated. The residue was taken up in a small amount of water (˜5 mL), filtered, and evaporated to yield 3.75 g (60%) of crude 2-(4-bromo-benzyl)-4-hydroxy-pyrrolidine-2-carboxylic acid. The crude material was used without further purification.

Sodium hydroxide (0.244 g, 6.1 mmol) was dissolved in THF/water (1/1, 30 mL) and the above carboxylic acid (3 mmol), and 3,5-dichlorophenyl isocyanate (553 mg, 3 mmol) were added successively. Stirring was continued for 6 h at room temperature, after which the solution was acidified to pH 2 with 2 N HCl and extracted with EtOAc three times.

The combined extracts were washed with water and brine and then dried over MgSO₄. The solvent was evaporated to yield 780 mg (53%) of urea intermediate.

The above urea (1.36 g, 2.8 mmol) was dissolved in DMF (30 mL) and 1-hydroxy-benzotriazole hydrate (565 mg, 4.17 mmol) and 1-(3-dimethylaminopropyl)-3-ethylcarbodiimide hydrochloride (800 mg, 4.17 mmol) were added successively. The solution was stirred for 6 h at room temperature after which triethylamine (370 μL, 2.2 mmol) was added. Stirring was continued overnight. The solution was diluted with saturated aqueous ammonium chloride solution and extracted with EtOAc three times. The combined extracts were washed with brine, dried over magnesium sulfate and evaporated. The residue was purified by column chromatography on silica gel to yield 305 mg (23%) of the title compound.

### Example 2

Isobutyric Acid 7a-(4-bromo-benzyl)-2-(3,5-dichloro-phenyl)-1,3-dioxo-hexahydro-pyrrolo[1,2-c]imidazol-6-yl Ester (Method A)

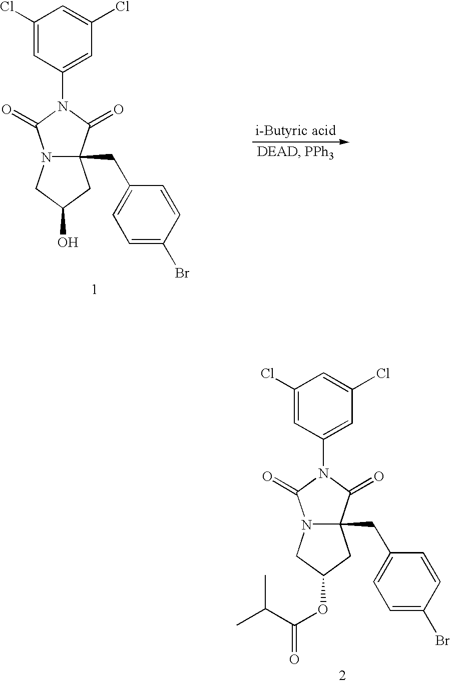

Compound 1 (Example 1) (23.5 mg, 0.05 mmol) was dissolved in THF (0.3 mL), and diethyl azodi-carboxylate (9.4 μL, 0.055 mmol) was added. After 5 min, isobutyric acid (5.0 μL, 0.055 mmol) and triphenylphosphine (14.4 mg, 0.055 mmol) were added and the mixture was stirred at ambient temperature for 19 h. The mixture was concentrated and the residue was purified by column chromatography on silica gel to yield 10 mg (37%) of the title compound.

### Example 3

3-[2-(3,5-Dichloro-phenyl)-1,3-dioxo-7a-(4-pyrimidin-5-yl-benzyl)-hexahydro-pyrrolo[1,2-c]imidazol-6-yloxycarbonylamino]-propionic Acid (Method B)

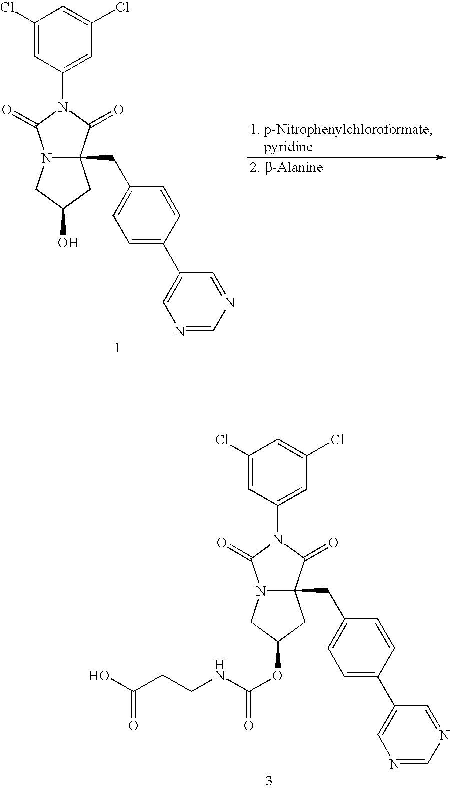

Compound 1 (Example 1) (75.5 mg, 0.16 mmol), pyridine (27 μl, 0.32 mmol) and 4-(dimethylamino)pyridine (catalytic) were dissolved in THF (2.0 mL) and the solution was cooled in an ice/water bath. 4-Nitrophenyl chloroformate (65 mg, 0.32 mmol) was added and the reaction was allowed to stir overnight at room temperature. The reaction mixture was then diluted with EtOAc, washed with saturated aqueous ammonium chloride solution, and dried over magnesium sulfate. The solution was filtered and evaporated. The crude 4-nitrophenyl carbonate was dissolved in MeOH/THF (1/1; 6 mL) and p-alanine (146 mg, 1.6 mmol) was added. The resulting solution was stirred for 2 h after which it was concentrated and then reconstituted with dichloromethane (20 mL). MP-Carbonate resin was added (125 mg, 4 mmol, 3.2 mmol/g) and the mixture was agitated for 3 h on an orbital shaker. The solution was filtered and the resin washed with dichloromethane. The filtrate was concentrated and purified by column chromatography on silica gel to yield 58 mg (62%) of the title compound.

The following additional compounds of the invention were prepared by methods analogous to those described above. Each of the compounds below was characterized by NMR and MS.

| Exam- | |
|---|---|
| ple | |
| No. | Structure |
| 4 | 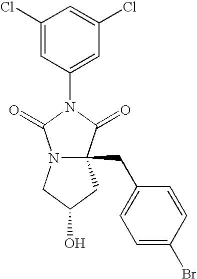 |
| 5 | 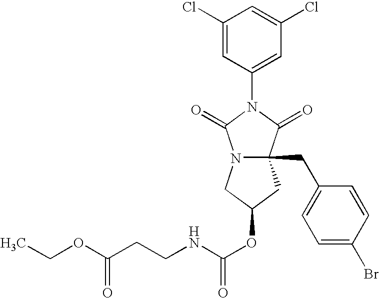 |
| 6 | 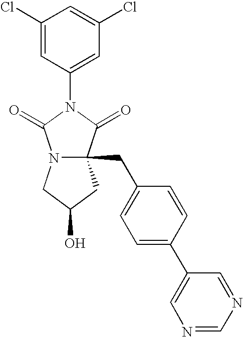 |
| 7 | 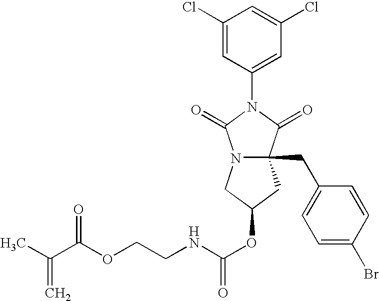 |
| 8 | 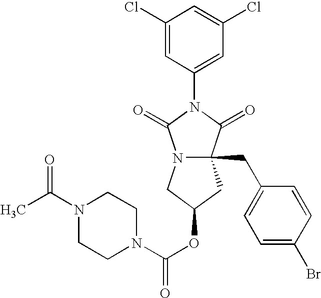 |
| 9 | 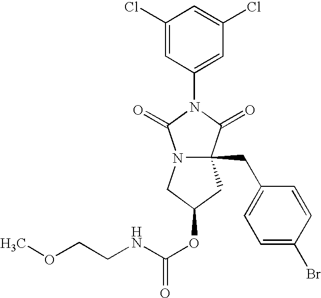 |
| 10 | 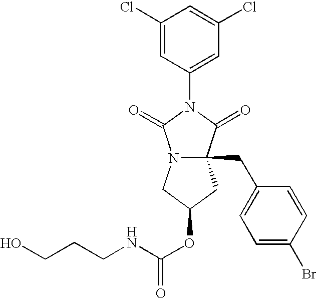 |
| 11 | 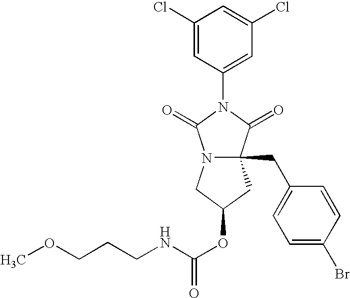 |
| 12 | 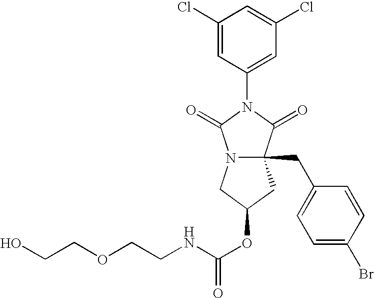 |
| 13 | 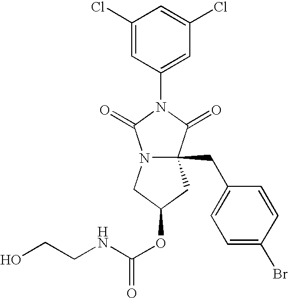 |
| 14 | 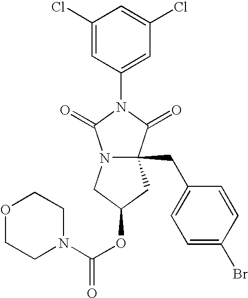 |
| 15 | 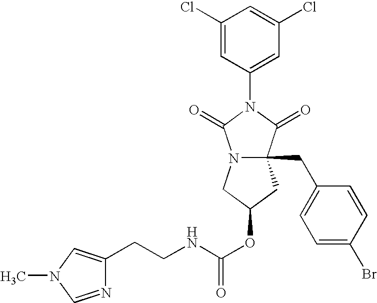 |
| 16 | 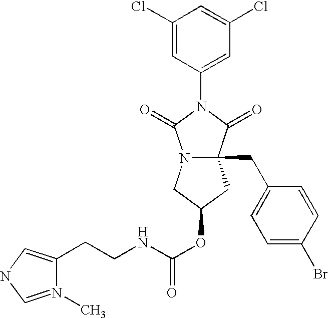 |
| 17 | 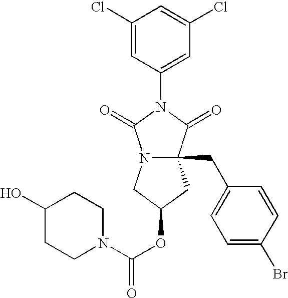 |
| 18 | 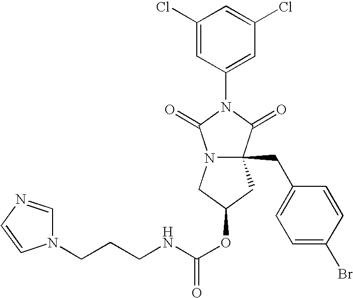 |
| 19 | 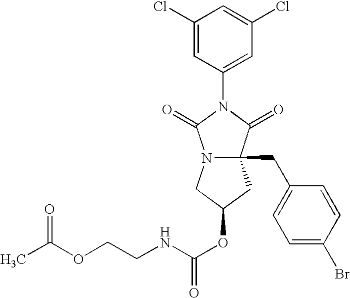 |
| 20 | 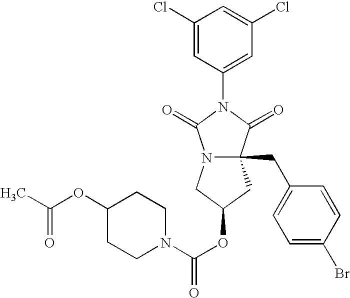 |
| 21 | 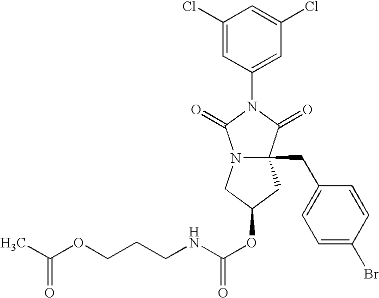 |
| 22 | 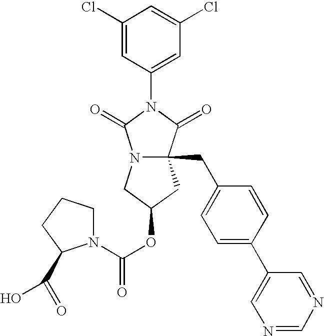 |
| 23 | 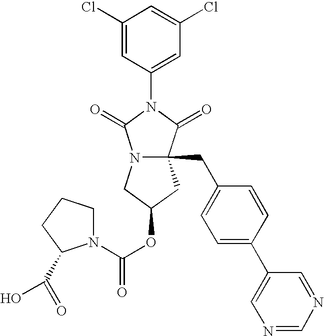 |
| 24 | 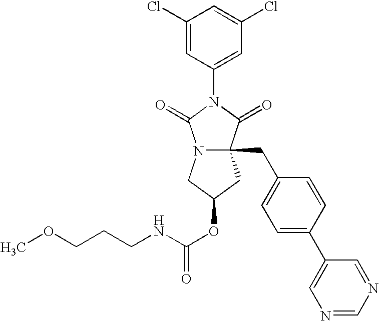 |
| 25 | 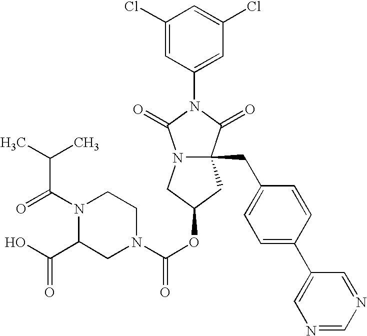 |

Description of Biological Properties

The biological properties of representative compounds of the formula I were investigated by way of the experimental protocol described below.

Assay to Determine Inhibition of LFA-1 Binding to ICAM-1

Purpose of Assay:

This assay protocol is designed to study the direct antagonism, by a test compound, of the interaction of the CAM, ICAM-1 with the Leukointegrin CD18/CD11a (LFA-1).

Description of Assay Protocol:

LFA-1 is immunopurified using the TS2/4 antibody from a 20 g pellet of human JY or SKW3 cells, utilizing a protocol previously described (Dustin, M. J.; et al., J. Immunol. 1992, 148, 2654-2660). The LFA-1 is purified from SKW3 lysates by immunoaffinity chromatography on TS2/4 LFA-1 mAb Sepharose and eluted at pH 11.5 in the presence of 2 mM MgCl₂ and 1% octylglucoside. After collection and neutralization of fractions from the TS2/4 column, samples are pooled and precleared with Protein G agarose.

A soluble form of ICAM-1 is constructed, expressed, purified and characterized as previously described (Marlin, S.; et al., Nature, 1990, 344, 70-72 and see Arruda, A.; et al., Antimicrob. Agents Chemother. 1992, 36, 1186-1192). Briefly, isoleucine 454 which is located at the putative boundary between domain 5 of the ectodomain and the transmembrane domain, is changed to a stop codon using standard oligonucleotide-directed mutagenesis. This construction yields a molecule identical with the first 453 amino acids of membrane bound ICAM-1. An expression vector is created with a hamster dihydrofolate reductase gene, a neomycin-resistance marker, and the coding region of the sICAM-1 construct described above, along with the promoter, splice signals, and polyadenylation signal of the SV40 early region. The recombinant plasmid is transfected into CHO DUX cells using standard calcium phosphate methods. Cells are passaged in selective media (G418) and colonies secreting sICAM-1 are amplified using methotrexate. sICAM-1 is purified from serum-free media using traditional non-affinity chromatographic techniques, including ion exchange and size exclusion chromatography.

LFA-1 binding to ICAM-1 is monitored by first incubating sICAM-1 at 40 μg/mL in Dulbecco's phosphate buffered saline with calcium and magnesium, additional 2 mM MgCl₂ and 0.1 mM PMSF (Diluting Buffer) in a 96-well plate for 30 min at room temperature. Plates are then blocked by the addition of 2% (w/v) bovine serum albumin in Diluting Buffer for 37° C. for 1 h. Blocking solution is removed from wells, and test compounds are diluted and then added followed by the addition of approximately 25 ng of immunoaffinity purified LFA-1. The LFA-1 is incubated in the presence of test compound and ICAM-1 at 37° C. for 1 h. Wells are washed 3 times with Diluting Buffer. The bound LFA-1 is detected by the addition of a polyclonal antibody directed against a peptide corresponding to the CD18 cytoplasmic tail in a 1:100 dilution with Diluting Buffer and 1% BSA and allowed to incubate for 45 min at 37° C. Wells are washed 3 times with Diluting Buffer and the bound polyclonal antibody is detected by the addition of a 1:4000 dilution of horse radish peroxidase conjugated to goat immunoglobulin directed against rabbit immunoglobulin. This reagent is allowed to incubate for 20 min at 37° C., wells are washed as above and the substrate for the horse radish peroxidase is added to each well to develop a quantitative colorimetric signal proportional to the amount of LFA-1 bound to sICAM-1. Soluble ICAM-1 (60 μg/mL) is used as a positive control for inhibition of the LFA-1/ICAM-1 interaction. The lack of the addition of LFA-1 to the binding assay is used as a background control for all samples. A dose-response curve is obtained for all test compounds.

All compounds made in the above examples were tested in this assay and each found to have a K_{d}<10 μM.

Description of Therapeutic Use

The novel small molecules of formula I provided by the invention inhibit the ICAM-1/LFA-1 dependent homotypic aggregation of human lymphocytes and human lymphocyte adherence to ICAM-1. These compounds have therapeutic utility in the modulation of immune cell activation/proliferation, e.g., as competitive inhibitors of intercellular ligand/receptor binding reactions involving CAMs and Leukointegrins. To be more specific, the compounds of the invention may be used to treat certain inflammatory conditions, including conditions resulting from a response of the non-specific immune system in a mammal (e.g., adult respiratory distress syndrome, shock, oxygen toxicity, multiple organ injury syndrome secondary to septicemia, multiple organ injury syndrome secondary to trauma, reperfusion injury of tissue due to cardiopulmonary bypass, myocardial infarction or use with thrombolysis agents, acute glomerulonephritis, vasculitis, reactive arthritis, dermatosis with acute inflammatory components, stroke, thermal injury, hemodialysis, leukapheresis, ulcerative colitis, necrotizing enterocolitis and granulocyte transfusion associated syndrome) and conditions resulting from a response of the specific immune system in a mammal (e.g., psoriasis, organ/tissue transplant rejection, graft vs. host reactions and autoimmune diseases including Raynaud's syndrome, autoimmune thyroiditis, dermatitis, multiple sclerosis, rheumatoid arthritis, insulin-dependent diabetes mellitus, uveitis, inflammatory bowel disease including Crohn's disease and ulcerative colitis, and systemic lupus erythematosus). The compounds of the invention may also be used in treating asthma or as an adjunct to minimize toxicity with cytokine therapy in the treatment of cancers. In general these compounds may be employed in the treatment of those diseases currently treatable through steroid therapy.

Thus, another aspect of the invention is the provision of a method for the treatment or prophylaxis of the above-described conditions through the adminstration of therapeutic or prophylactic amounts of one or more compounds of the formula I.

In accordance with the method provided by the invention, the novel compounds of formula I may be administered for either a prophylactic or therapeutic purpose either alone or with other immunosuppressive or antiinflammatory agents. When provided prophylactically, the immunosuppressive compound(s) are provided in advance of any inflammatory response or symptom (for example, prior to, at, or shortly after the time of an organ or tissue transplant but in advance of any symptoms of organ rejection). The prophylactic administration of a compound of the formula I serves to prevent or attenuate any subsequent inflammatory response (such as, for example, rejection of a transplanted organ or tissue, etc.). The therapeutic administration of a compound of the formula I serves to attenuate any actual inflammation (such as, for example, the rejection of a transplanted organ or tissue). Thus, in accordance with the invention, a compound of the formula I can be administered either prior to the onset of inflammation (so as to suppress an anticipated inflammation) or after the initiation of inflammation.

The novel compounds of the formula I may, in accordance with the invention, be administered in single or divided doses by the oral, parenteral or topical routes. A suitable oral dosage for a compound of formula I would be in the range of about 0.1 mg to 10 g per day. In parenteral formulations, a suitable dosage unit may contain from 0.1 to 250 mg of said compounds, whereas for topical administration, formulations containing 0.01 to 1% active ingredient are preferred. It should be understood, however, that the dosage administration from patient to patient will vary and the dosage for any particular patient will depend upon the clinician's judgement, who will use as criteria for fixing a proper dosage the size and condition of the patient as well as the patient's response to the drug.

When the compounds of the present invention are to be administered by the oral route, they may be administered as medicaments in the form of pharmaceutical preparations which contain them in association with a compatible pharmaceutical carrier material. Such carrier material can be an inert organic or inorganic carrier material suitable for oral administration. Examples of such carrier materials are water, gelatin, talc, starch, magnesium stearate, gum arabic, vegetable oils, polyalkylene-glycols, petroleum jelly and the like.

The pharmaceutical preparations can be prepared in a conventional manner and finished dosage forms can be solid dosage forms, for example, tablets, dragees, capsules, and the like, or liquid dosage forms, for example solutions, suspensions, emulsions and the like. The pharmaceutical preparations may be subjected to conventional pharmaceutical operations such as sterilization. Further, the pharmaceutical preparations may contain conventional adjuvants such as preservatives, stabilizers, emulsifiers, flavor-improvers, wetting agents, buffers, salts for varying the osmotic pressure and the like. Solid carrier material which can be used include, for example, starch, lactose, mannitol, methyl cellulose, microcrystalline cellulose, talc, silica, dibasic calcium phosphate, and high molecular weight polymers (such as polyethylene glycol).

For parenteral use, a compound of formula I can be administered in an aqueous or non-aqueous solution, suspension or emulsion in a pharmaceutically acceptable oil or a mixture of liquids, which may contain bacteriostatic agents, antioxidants, preservatives, buffers or other solutes to render the solution isotonic with the blood, thickening agents, suspending agents or other pharmaceutically acceptable additives. Additives of this type include, for example, tartrate, citrate and acetate buffers, ethanol, propylene glycol, polyethylene glycol, complex formers (such as EDTA), antioxidants (such as sodium bisulfite, sodium metabisulfite, and ascorbic acid), high molecular weight polymers (such as liquid polyethylene oxides) for viscosity regulation and polyethylene derivatives of sorbitol anhydrides. Preservatives may also be added if necessary, such as benzoic acid, methyl or propyl paraben, benzalkonium chloride and other quaternary ammonium compounds.

The compounds of this invention may also be administered as solutions for nasal application and may contain in addition to the compounds of this invention suitable buffers, tonicity adjusters, microbial preservatives, antioxidants and viscosity-increasing agents in an aqueous vehicle. Examples of agents used to increase viscosity are polyvinyl alcohol, cellulose derivatives, polyvinylpyrrolidone, polysorbates or glycerin. Microbial preservatives added may include benzalkonium chloride, thimerosal, chloro-butanol or phenylethyl alcohol.

Additionally, the compounds provided by the invention can be administered topically or by suppository.

Formulations

Compounds of the formula I can be formulated for therapeutic administration in a number of ways. Descriptions of several exemplary formulations are given below.

Capsules or Tablets

| Example A-1 | | Example A-2 | |
|---|---|---|---|
| Ingredients | Quantity | Ingredients | Quantity |
| Compound of formula I | 250 mg | Compound of | 50 mg |
| | | formula I | |
| Starch | 160 mg | Dicalcium Phosphate | 160 mg |
| Microcrys. Cellulose | 90 mg | Microcrys. Cellulose | 90 mg |
| Sodium Starch Glycolate | 10 mg | Stearic acid | 5 mg |
| Magnesium Stearate | 2 mg | Sodium Starch | 10 mg |
| | | Glycolate | |
| Fumed colloidal silica | 1 mg | Fumed colloidal silica | 1 mg |

The compound of formula I is blended into a powder mixture with the premixed excipient materials as identified above with the exception of the lubricant. The lubricant is then blended in and the resulting blend compressed into tablets or filled into hard gelatin capsules.

Parenteral Solutions

| Ingredients | Quantity |
|---|---|
| Compound of formula I | 500 mg |
| PEG 400 | 40% by volume |
| Ethyl Alcohol | 5% by volume |
| Saline | 55% by volume |

The excipient materials are mixed and then added to one of the compounds of formula I in such volume as is necessary for dissolution. Mixing is continued until the solution is clear. The solution then filtered into the appropriate vials or ampoules and sterilized by autoclaving.

Suspension

| | |
|---|---|
| Ingredients | Quantity |

| | | |
|---|---|---|
| Compound of formula I | 100 | mg |
| Citric acid | 1.92 | g |
| Benzalkonium chloride | 0.025% | by weight |
| EDTA | 0.1% | by weight |
| Polyvinylalcohol | 10% | by weight |

| | |
|---|---|
| Water | q.s. to 100 mL |

The excipient materials are mixed with the water and thereafter one of the compounds of formula I is added and mixing is continued until the suspension is homogeneous. The suspension is then transferred into the appropriate vials or ampoules.

Topical Formulation

| | |
|---|---|
| Ingredients | Quantity |

| | | |
|---|---|---|
| Compound of formula I | 5% | by weight |
| Tefose 63 | 13% | by weight |
| Labrafil M 1944 CS | 3% | by weight |
| Paraffin Oil | 8% | by weight |
| Methylparaben (MP) | 0.15% | by weight |
| Propylparaben (PP) | 0.05% | by weight |

| | |
|---|---|
| Deionized water | q.s. to 100 |

The proper amounts of Tefose 63, Labrafil M 1944 CS, Paraffin oil and water are mixed and heated at 75° C. until all components have melted. The mixture is then cooled to 50° C. with continuous stirring. Methylparaben and propylparaben are added with mixing and the mixture is cooled to ambient temperature. The compound of formula I is added to the mixture and blended well.

1. A compound of the formula I 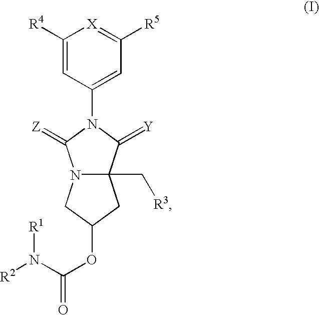
wherein:
Y is an oxygen or sulfur atom;
Z is an oxygen or sulfur atom;
R¹ is selected from the class consisting of:
branched or unbranched alkyl of 1 to 10 carbon atoms, alkenyl of 2 to 10 carbon atoms or cycloalkyl or cycloalkenyl of 3 to 6 carbon atoms, wherein one to three carbon atoms in said alkyl, alkenyl, cycloalkyl or cycloalkenyl may be replaced by —O—, —S—, —S(O)—, —S(O)₂—, —NH— or —N(CH₃)—, and in which said alkyl, alkenyl, cycloalkyl or cycloalkenyl group one to six hydrogen atoms are optionally and independently replaced with:
(i) halogen,
(ii) oxo,
(iii) aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzothiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein one or more hydrogen atoms of said aryl or heteroaryl group are optionally and independently replaced with:
(a) alkyl of 1 to 3 carbon atoms,
(b) —COOH,
(c) a group of the formula COOR⁶, wherein R⁶ is straight or branched alkyl of 1 to 5 carbon atoms or cycloalkyl of 3 to 5 carbon atoms,
(d) a group of the formula —NR⁷R⁸, wherein R⁷ and R⁸ are each independently a hydrogen atom, alkyl of 1 to 6 carbon atoms, cycloalkyl of 3 to 6 carbon atoms or acyl of 1 to 7 carbon atoms, or wherein R⁷ and R⁸ constitute a saturated hydrocarbon bridge of 3 to 5 carbon atoms which together with the nitrogen atom between them form a heterocyclic ring,
(e) a group of the formula —CONR⁹R¹⁰, wherein R⁹ and R¹⁰ are each independently a hydrogen atom, alkyl of 1 to 6 carbon atoms or cycloalkyl of 3 to 6 carbon atoms, or wherein R⁹ and R¹⁰ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,
(f) a group of the formula —OR¹¹, wherein R¹¹ is a hydrogen atom, or an alkyl or acyl group of 1 to 7 carbon atoms,
(g) a group of the formula —SR¹², wherein R¹² is a hydrogen atom, or an alkyl or acyl group of 1 to 7 carbon atoms,
(h) —CN,
(i) an amidino group of the formula 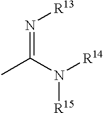
wherein R¹³, R¹⁴ and R¹⁵ are each, independently, a hydrogen atom or alkyl of 1 to 3 carbon atoms and wherein two of R¹³, R¹⁴ and R¹⁵ may additionally constitute a saturated hydrocarbon bridge of 3 to 5 carbon atoms which together with the nitrogen atom(s) between them form a heterocyclic ring,
(j) halogen,
(k) a group of the formula —NHCONHalkyl, wherein the alkyl moiety contains 1 to 3 carbon atoms, or
(l) a group of the formula —NHCOOalkyl, wherein the alkyl moiety contains 1 to 3 carbon atoms,
(iv) a group of the formula —COOR¹⁶, wherein R¹⁶ is hydrogen or straight or branched alkyl of 1 to 7 carbon atoms or cycloalkyl of 3 to 6 carbon atoms,
(v) —CN,
(vi) a group of the formula —CONR¹⁷R¹⁸, wherein R¹⁷ and R¹⁸ are each, independently, a hydrogen atom, alkyl of 1 to 6 carbon atoms or cycloalkyl of 3 to 6 carbon atoms, or wherein R¹⁷ and R¹⁸ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,
(vii) a group of the formula —OR¹⁹, wherein R¹⁹ is a hydrogen atom, or a straight or branched alkyl or acyl group of 1 to 7 carbon atoms, wherein one or more hydrogen atoms of said alkyl or acyl group are optionally replaced with a group independently selected from the class consisting of —OH, —Oalkyl (wherein the alkyl moiety contains 1 to 6 carbon atoms), —NH₂, —NHMe and —NMe₂,
(viii) a group of the formula —SR²⁰, wherein R²⁰ is a hydrogen atom, or an alkyl or acyl group of 1 to 7 carbon atoms, wherein one or more hydrogen atoms of said alkyl or acyl group are optionally replaced with a group independently selected from the class consisting of —OH, —Oalkyl (wherein the alkyl moiety is 1 to 6 carbon atoms), —NH₂, —NHMe and —NMe₂,
(ix) a group of the formula —NR²¹R²², wherein R²¹ and R²² are each, independently,
(a) a hydrogen atom,
(b) straight or branched alkyl or acyl of 1 to 7 carbon atoms or cycloalkyl of 3 to 7 carbon atoms, wherein said one or more hydrogen atoms of said alkyl or acyl group are optionally replaced with a group independently selected from the class consisting of —OH, —Oalkyl (wherein the alkyl moiety is 1 to 6 carbon atoms), —NH₂, —NHMe and —NMe₂,
(c) a group of the formula —(CH₂)ₘCOOH, wherein m is 0, 1 or 2,
(d) a group of the formula —(CH₂)ₙCOOR²³, wherein n is 0, 1 or 2, and wherein R²³ is straight or branched alkyl of 1 to 6 carbon atoms, or
(e) a group of the formula —(CH₂)ₙCONHR²⁴, wherein n is 0, 1 or 2, and wherein R²⁴ is straight or branched alkyl of 1 to 6 carbon atoms,
(x) a saturated, or partially unsaturated heterocyclic group selected from imidazolinyl, imidazolidinyl, pyrrolinyl, pyrrolidinyl, piperidinyl, piperazinyl, morpholinyl, thiomorpholinyl, thiazolidinyl, azepinyl, tetrahydropyranyl, tetrahydrofuranyl, benzodioxolyl, tetrahydrothiophenyl and sulfolanyl, wherein said heterocyclic group is optionally mono- or polysubstituted with:
(a) alkyl of 1 to 3 carbon atoms,
(b) —COOH,
(g) a group of the formula —COOR⁶, wherein R⁶ is straight or branched alkyl of 1 to 5 carbon atoms or cycloalkyl of 3 to 5 carbon atoms,
(h) a group of the formula —C(O)R⁶, wherein R⁶ is straight or branched alkyl of 1 to 5 carbon atoms or cycloalkyl of 3 to 5 carbon atoms,
(e) a group of the formula —NR⁷R⁸, wherein R⁷ and R⁸ are each independently a hydrogen atom, alkyl of 1 to 6 carbon atoms, cycloalkyl of 3 to 6 carbon atoms or acyl of 1 to 7 carbon atoms, or wherein R⁷ and R⁸ constitute a saturated hydrocarbon bridge of 3 to 5 carbon atoms which together with the nitrogen atom between them form a heterocyclic ring,
(f) a group of the formula —CONR⁹R¹⁰, wherein R⁹ and R¹⁰ are each independently a hydrogen atom, alkyl of 1 to 6 carbon atoms or cycloalkyl of 3 to 6 carbon atoms, or wherein R⁹ and R¹⁰ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,
(g) a group of the formula —OR¹¹, wherein R¹¹ is a hydrogen atom, or an alkyl or acyl group of 1 to 7 carbon atoms,
(h) a group of the formula —SR¹², wherein R¹² is a hydrogen atom, or an alkyl or acyl group of 1 to 7 carbon atoms,
(i) —CN,
(j) an amidino group of the formula 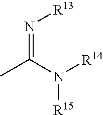
wherein R¹³, R¹⁴ and R¹⁵ are each, independently, a hydrogen atom or alkyl of 1 to 3 carbon atoms and wherein two of R¹³, R¹⁴ and R¹⁵ may additionally constitute a saturated hydrocarbon bridge of 3 to 5 carbon atoms which together with the nitrogen atom(s) between them form a heterocyclic ring,
(k) halogen,
(l) a group of the formula —NHCONHalkyl, wherein the alkyl moiety contains 1 to 3 carbon atoms, or
(m) halogen,
(n) a group of the formula —NHCONHalkyl, wherein the alkyl moiety contains 1 to 3 carbon atoms,
(o) a group of the formula —NHCOOalkyl, wherein the alkyl moiety contains 1 to 3 carbon atoms, or
(p) oxo
or
(xii) a cycloalkyl group of 3 to 7 carbon atoms;
R² is H or C₁₋₃alkyl; or
R¹ and R² together with the nitrogen they are bonded to may form a heterocyclic ring selected from the class consisting of piperidine, pyrrolidine, piperazine, morpholine and thiomorpholine, wherein said heterocyclic ring is optionally mono-, di- or trisubstituted with:
(a) alkyl of 1 to 3 carbon atoms,
(b) —COOH,
(ci) a group of the formula —COOR⁶, wherein R⁶ is straight or branched alkyl of 1 to 5 carbon atoms or cycloalkyl of 3 to 5 carbon atoms,
(dii) a group of the formula —C(O)R⁶, wherein R⁶ is straight or branched alkyl of 1 to 5 carbon atoms or cycloalkyl of 3 to 5 carbon atoms,
(e) a group of the formula —NR⁷R⁸, wherein R⁷ and R⁸ are each independently a hydrogen atom, alkyl of 1 to 6 carbon atoms, cycloalkyl of 3 to 6 carbon atoms or acyl of 1 to 7 carbon atoms, or wherein R⁷ and R⁸ constitute a saturated hydrocarbon bridge of 3 to 5 carbon atoms which together with the nitrogen atom between them form a heterocyclic ring,
(f) a group of the formula —CONR⁹R¹⁰, wherein R⁹ and R¹⁰ are each independently a hydrogen atom, alkyl of 1 to 6 carbon atoms or cycloalkyl of 3 to 6 carbon atoms, or wherein R⁹ and R¹⁰ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,
(g) a group of the formula —OR¹¹, wherein R¹¹ is a hydrogen atom, or an alkyl or acyl group of 1 to 7 carbon atoms,
(h) a group of the formula —SR¹², wherein R¹² is a hydrogen atom, or an alkyl or acyl group of 1 to 7 carbon atoms,
(i) —CN,
(j) an amidino group of the formula 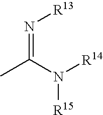
wherein R¹³, R¹⁴ and R¹⁵ are each, independently, a hydrogen atom or alkyl of 1 to 3 carbon atoms and wherein two of R¹³, R¹⁴ and R¹⁵ may additionally constitute a saturated hydrocarbon bridge of 3 to 5 carbon atoms which together with the nitrogen atom(s) between them form a heterocyclic ring,
(k) halogen,
(l) a group of the formula —NHCONHalkyl, wherein the alkyl moiety contains 1 to 3 carbon atoms, or
(m) a group of the formula —NHCONHalkyl, wherein the alkyl moiety contains 1 to 3 carbon atoms,
(n) a group of the formula —NHCOOalkyl, wherein the alkyl moiety contains 1 to 3 carbon atoms, or
(o) oxo
R³ is
aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzothiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein one or more of the hydrogen atoms of said aryl or heteroaryl group is optionally and independently replaced with:
(A) R⁴⁴, which is aryl or heteroaryl selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzothiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein one or more of the hydrogen atoms of said aryl or heteroaryl group is optionally and independently replaced with:
(i) branched or unbranched alkyl of 1 to 6 carbon atoms or cycloalkyl of 3 to 6 carbon atoms, which alkyl or cycloakyl group is optionally mono- or polysubstituted with halogen or oxo,
(ii) a group of the formula —COOR⁴⁵, wherein R⁴⁵ is straight or branched alkyl of 1 to 5 carbon atoms or cycloalkyl of 3 to 5 carbon atoms,
(iii) a group of the formula —NR⁴⁶R⁴⁷, wherein R⁴⁶ and R⁴⁷ are each, independently, a hydrogen atom, alkyl or fluoroalkyl of 1 to 6 carbon atoms, cycloalkyl of 3 to 6 carbon atoms or acyl of 1 to 7 carbon atoms, or wherein R⁴⁶ and R⁴⁷ constitute a saturated hydrocarbon bridge of 3 to 5 carbon atoms which together with the nitrogen atom between them form a heterocyclic ring,
(iv) a group of the formula —CONR⁴⁸R⁴⁹, wherein R⁴⁸ and R⁴⁹ are each, independently, a hydrogen atom, alkyl or fluoroalkyl of 1 to 6 carbon atoms or cycloalkyl of 3 to 6 carbon atoms, or wherein R⁴⁸ and R⁴⁹ constitute a saturated hydrocarbon bridge of 3 to 5 carbon atoms which together with the nitrogen atom between them form a heterocyclic ring,
(v) a group of the formula —OR⁵⁰, wherein R⁵⁰ is a hydrogen atom, or an alkyl, fluoroalkyl or acyl group of 1 to 7 carbon atoms,
(vi) a group of the formula —SR⁵¹, wherein R⁵¹ is a hydrogen atom, or an alkyl, fluoroalkyl or acyl group of 1 to 7 carbon atoms,
(vii) —CN,
(viii) nitro, or
(ix) halogen,
(B) methyl, which is optionally mono- or polysubstituted with fluorine atoms and additionally is optionally monosubstituted with R⁴⁴,
(C) branched or unbranched alkyl of 2 to 6 carbon atoms or cycloalkyl of 3 to 6 carbon atoms, which alkyl or cycloakyl group is optionally mono- or polysubstituted with halogen or oxo,
(D) a group of the formula —COOR⁵², wherein R⁵² is straight or branched alkyl of 1 to 5 carbon atoms or cycloalkyl of 3 to 5 carbon atoms,
(E) a group of the formula —NR⁵³R⁵⁴, wherein R⁵³ and R⁵⁴ are each, independently, a hydrogen atom, alkyl or fluoroalkyl of 1 to 6 carbon atoms, cycloalkyl of 3 to 6 carbon atoms or acyl of 1 to 7 carbon atoms, or wherein R⁵³ and R⁵⁴ constitute a saturated hydrocarbon bridge of 3 to 5 carbon atoms which together with the nitrogen atom between them form a heterocyclic ring, and wherein one of R⁵³ and R⁵⁴ may additionally be the group R⁴⁴,
(F) a group of the formula —CONR⁵⁵R⁵⁶, wherein R⁵⁵ and R⁵⁶ are each, independently, a hydrogen atom, alkyl or fluoroalkyl of 1 to 6 carbon atoms or cycloalkyl of 3 to 6 carbon atoms, or wherein R⁵⁵ and R⁵⁶ constitute a saturated hydrocarbon bridge of 3 to 5 carbon atoms which together with the nitrogen atom between them form a heterocyclic ring, and wherein one of R⁵⁵ and R⁵⁶ may additionally be the group R⁴⁴,
(G) a group of the formula —COR⁵⁷, wherein R⁵⁷ is a hydrogen atom, straight or branched alkyl of 1 to 5 carbon atoms, cycloalkyl of 3 to 5 carbon atoms or R⁴⁴,
(H) a group of the formula —OR⁵⁸, wherein R⁵⁸ is a hydrogen atom, an alkyl, fluoroalkyl or acyl group of 1 to 7 carbon atoms, or R⁴⁴,
(I) a group of the formula —SR⁵⁹, wherein R⁵⁹ is a hydrogen atom, an alkyl, fluoroalkyl or acyl group of 1 to 7 carbon atoms, or R⁴⁴,
(J) —CN,
(K) nitro, or
(L) halogen;
R⁴ is Cl or trifluoromethyl;
X is ═N— or ═C(R⁶⁰)— wherein R⁶⁰ is a hydrogen, fluorine, chlorine, bromine or iodine atom, methyl or trifluoromethyl; and,
R⁵ is a hydrogen, fluorine, chlorine, bromine or iodine atom, methyl, —CN, nitro or trifluoromethyl, with the condition that when X is ═N— or ═C(H)—, R⁵ is chlorine, trifluoromethyl, —CN or nitro;
or a pharmaceutically acceptable salt thereof.

2. A compound of the formula I, as set forth in claim 1, wherein:
Y is an oxygen atom;
Z is an oxygen atom;
R¹ is selected from the class consisting of:
branched or unbranched alkyl of 1 to 10 carbon atoms or alkenyl of 2 to 10 carbon atoms, wherein one to three carbon atoms in said alkyl, alkenyl, cycloalkyl or cycloalkenyl may be replaced by —O—, —NH— or —N(CH₃)—, and in which said alkyl or alkenyl group one to six hydrogen atoms are optionally and independently replaced with:
(i) halogen,
(ii) oxo,
(vi) aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, pyrazinyl, isoindolyl, benzo[b]furanyl, benzo[b]thiophenyl, benzothiazolyl, benzimidazolyl, quinolinyl isoquinolinyl and quinazolinyl, wherein one or more hydrogen atoms of said aryl or heteroaryl group are optionally and independently replaced with:
(a) alkyl of 1 to 3 carbon atoms,
(b) —COOH,
(c) a group of the formula —COOR⁶, wherein R⁶ is straight or branched alkyl of 1 to 5 carbon atoms or cycloalkyl of 3 to 5 carbon atoms,
(d) a group of the formula —NR⁷R⁸, wherein R⁷ and R⁸ are each independently a hydrogen atom, alkyl of 1 to 6 carbon atoms, cycloalkyl of 3 to 6 carbon atoms or acyl of 1 to 7 carbon atoms, or wherein R⁷ and R⁸ constitute a saturated hydrocarbon bridge of 3 to 5 carbon atoms which together with the nitrogen atom between them form a heterocyclic ring,
(e) a group of the formula —CONR⁹R¹⁰, wherein R⁹ and R¹⁰ are each independently a hydrogen atom, alkyl of 1 to 6 carbon atoms or cycloalkyl of 3 to 6 carbon atoms, or wherein R⁹ and R¹⁰ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,
(f) a group of the formula —OR¹¹, wherein R¹¹ is a hydrogen atom, or an alkyl or acyl group of 1 to 7 carbon atoms,
(g) a group of the formula —SR¹², wherein R¹² is a hydrogen atom, or an alkyl or acyl group of 1 to 7 carbon atoms,
(h) —CN,
(i) halogen,
(k) a group of the formula —NHCONHalkyl, wherein the alkyl moiety contains 1 to 3 carbon atoms, or
(l) a group of the formula —NHCOOalkyl, wherein the alkyl moiety contains 1 to 3 carbon atoms,
(iv) —CN,
(v) a group of the formula —OR¹⁹, wherein R¹⁹ is a hydrogen atom, or a straight or branched alkyl or acyl group of 1 to 7 carbon atoms, wherein one or more hydrogen atoms of said alkyl or acyl group are optionally replaced with a group independently selected from the class consisting of —OH, —Oalkyl (wherein the alkyl moiety contains 1 to 6 carbon atoms), —NH₂, —NHMe and —NMe₂,
(vi) a group of the formula —SR²⁰, wherein R²⁰ is a hydrogen atom, or an alkyl or acyl group of 1 to 7 carbon atoms, wherein one or more hydrogen atoms of said alkyl or acyl group are optionally replaced with a group independently selected from the class consisting of —OH, —Oalkyl (wherein the alkyl moiety is 1 to 6 carbon atoms), —NH₂, —NHMe and —NMe₂,
(vii) a group of the formula —NR²¹R²², wherein R²¹ and R²² are each, independently,
(a) a hydrogen atom,
(b) straight or branched alkyl or acyl of 1 to 7 carbon atoms or cycloalkyl of 3 to 7 carbon atoms, wherein said one or more hydrogen atoms of said alkyl or acyl group are optionally replaced with a group independently selected from the class consisting of —OH, —Oalkyl (wherein the alkyl moiety is 1 to 6 carbon atoms), —NH₂, —NHMe and —NMe₂,
(x) a saturated, or partially unsaturated heterocyclic group selected from imidazolinyl, imidazolidinyl, pyrrolinyl, pyrrolidinyl, piperidinyl, piperazinyl, morpholinyl, thiomorpholinyl, thiazolidinyl, azepinyl, tetrahydropyranyl, tetrahydrofuranyl, benzodioxolyl, tetrahydrothiophenyl and sulfolanyl, wherein said heterocyclic group is optionally mono- or polysubstituted with:
(a) alkyl of 1 to 3 carbon atoms,
(b) —COOH,
(i) a group of the formula —COOR⁶, wherein R⁶ is straight or branched alkyl of 1 to 5 carbon atoms or cycloalkyl of 3 to 5 carbon atoms,
(j) a group of the formula —C(O)R⁶, wherein R⁶ is straight or branched alkyl of 1 to 5 carbon atoms or cycloalkyl of 3 to 5 carbon atoms,
(e) a group of the formula —NR⁷R⁸, wherein R⁷ and R⁸ are each independently a hydrogen atom, alkyl of 1 to 6 carbon atoms, cycloalkyl of 3 to 6 carbon atoms or acyl of 1 to 7 carbon atoms, or wherein R⁷ and R⁸ constitute a saturated hydrocarbon bridge of 3 to 5 carbon atoms which together with the nitrogen atom between them form a heterocyclic ring,
(f) a group of the formula —CONR⁹R¹⁰, wherein R⁹ and R¹⁰ are each independently a hydrogen atom, alkyl of 1 to 6 carbon atoms or cycloalkyl of 3 to 6 carbon atoms, or wherein R⁹ and R¹⁰ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,
(g) a group of the formula —OR¹¹, wherein R¹¹ is a hydrogen atom, or an alkyl or acyl group of 1 to 7 carbon atoms,
(h) a group of the formula —SR¹², wherein R¹² is a hydrogen atom, or an alkyl or acyl group of 1 to 7 carbon atoms,
(i) —CN,
(j) halogen,
(k) a group of the formula —NHCONHalkyl, wherein the alkyl moiety contains 1 to 3 carbon atoms, or
(l) halogen,
(m) a group of the formula —NHCONHalkyl, wherein the alkyl moiety contains 1 to 3 carbon atoms,
(n) a group of the formula —NHCOOalkyl, wherein the alkyl moiety contains 1 to 3 carbon atoms, or
(o) oxo or
(diii) a cycloalkyl group of 3 to 7 carbon atoms;
R² is H; or
R¹ and R² together with the nitrogen they are bonded to may form a heterocyclic ring selected from the class consisting of piperidine, pyrrolidine, piperazine, morpholine and thiomorpholine, wherein said heterocyclic ring is optionally mono-, di- or trisubstituted with:
(a) alkyl of 1 to 3 carbon atoms,
(b) —COOH,
(c) a group of the formula —COOR⁶, wherein R⁶ is straight or branched alkyl of 1 to 5 carbon atoms or cycloalkyl of 3 to 5 carbon atoms,
(di) a group of the formula —C(O)R⁶, wherein R⁶ is straight or branched alkyl of 1 to 5 carbon atoms or cycloalkyl of 3 to 5 carbon atoms,
(h) a group of the formula —NR⁷R⁸, wherein R⁷ and R⁸ are each independently a hydrogen atom, alkyl of 1 to 6 carbon atoms, cycloalkyl of 3 to 6 carbon atoms or acyl of 1 to 7 carbon atoms,
(i) a group of the formula —CONR⁹R¹⁰, wherein R⁹ and R¹⁰ are each independently a hydrogen atom, alkyl of 1 to 6 carbon atoms or cycloalkyl of 3 to 6 carbon atoms
(j) a group of the formula —OR¹¹, wherein R¹¹ is a hydrogen atom, or an alkyl or acyl group of 1 to 7 carbon atoms,
(h) —CN,
(i) halogen,
R³ is
aryl or heteroaryl which is selected from the class consisting of phenyl, pyridyl and pyrimidinyl, wherein one or more of the hydrogen atoms of said aryl or heteroaryl group is optionally and independently replaced with:
(A) R⁴⁴, which is aryl or heteroaryl selected from the class consisting of phenyl, thiophenyl, pyridyl, pyrimidinyl, furyl, pyrrolyl, oxazolyl and thiazolyl, wherein one or more of the hydrogen atoms of said aryl or heteroaryl group is optionally and independently replaced with:
(i) branched or unbranched alkyl of 1 to 6 carbon atoms or cycloalkyl of 3 to 6 carbon atoms, which alkyl or cycloakyl group is optionally mono- or polysubstituted with halogen or oxo,
(ii) —CN,
(iii) nitro, or
(iv) halogen,
(B) methyl, which is optionally mono- or polysubstituted with fluorine atoms and additionally is optionally monosubstituted with R⁴⁴,
(C) branched or unbranched alkyl of 2 to 6 carbon atoms or cycloalkyl of 3 to 6 carbon atoms, which alkyl or cycloakyl group is optionally mono- or polysubstituted with halogen or oxo,
(D) a group of the formula —COOR⁵², wherein R⁵² is straight or branched alkyl of 1 to 5 carbon atoms or cycloalkyl of 3 to 5 carbon atoms,
(E) a group of the formula —COR⁵⁷, wherein R⁵⁷ is a hydrogen atom, straight or branched alkyl of 1 to 5 carbon atoms, cycloalkyl of 3 to 5 carbon atoms or R⁴⁴,
(F) a group of the formula —OR⁵⁸, wherein R⁵⁸ is a hydrogen atom, an alkyl, fluoroalkyl or acyl group of 1 to 7 carbon atoms, or R⁴⁴,
(G) —CN,
(H) nitro, or
(I) halogen;
R⁴ is Cl or trifluoromethyl;
X is ═N— or ═C(R⁶⁰)— wherein R⁶⁰ is a hydrogen, fluorine, chlorine, bromine or iodine atom, methyl or trifluoromethyl; and,
R⁵ is a fluorine, chlorine, bromine or iodine atom, methyl, —CN, nitro or trifluoromethyl, with the condition that when X is ═N— or ═C(H)—, R⁵ is chlorine, trifluoromethyl, —CN or nitro;
or a pharmaceutically acceptable salt thereof.

3. A compound of the formula I, as set forth in claim 1, wherein:
Y is an oxygen atom;
Z is an oxygen atom;
R¹ is selected from the class consisting of:
branched or unbranched alkyl of 1 to 10 carbon atoms or alkenyl of 2 to 10 carbon atoms, wherein one to three carbon atoms in said alkyl, alkenyl, cycloalkyl or cycloalkenyl may be replaced by —O—, —NH— or —N(CH₃)—, and in which said alkyl or alkenyl group one to six hydrogen atoms are optionally and independently replaced with:
(i) halogen,
(ii) oxo,
(vii) aryl or heteroaryl which is selected from the class consisting of phenyl, indolyl, pyridyl, pyrimidinyl, pyrrolyl, oxazolyl, thiazolyl, pyrazolyl, imidazolyl, pyrazinyl, benzothiazolyl, benzimidazolyl, quinolinyl isoquinolinyl and quinazolinyl, wherein one or more hydrogen atoms of said aryl or heteroaryl group are optionally and independently replaced with:
(a) alkyl of 1 to 3 carbon atoms,
(b) —COOH,
(ci) a group of the formula —NR⁷R⁸, wherein R⁷ and R⁸ are each independently a hydrogen atom or a methyl group,
(d) a group of the formula —CONR⁹R¹⁰, wherein R⁹ and R¹⁰ are each independently a hydrogen atom or a methyl group,
(e) a group of the formula —OR¹¹, wherein R¹¹ is a hydrogen atom, or an alkyl or acyl group of 1 to 7 carbon atoms,
(f) —CN,
(i) halogen,
(iv) —CN,
(v) a group of the formula —OR¹⁹, wherein R¹⁹ is a hydrogen atom, or a straight or branched alkyl or acyl group of 1 to 7 carbon atoms, wherein one or more hydrogen atoms of said alkyl or acyl group are optionally replaced with a group independently selected from the class consisting of —OH, —Oalkyl (wherein the alkyl moiety contains 1 to 6 carbon atoms), —NH₂, —NHMe and —NMe₂,
(vi) a group of the formula —NR²¹R²², wherein R²¹ and R²² are each, independently,
(a) a hydrogen atom,
(b) methyl
(x) a saturated, or partially unsaturated heterocyclic group selected from imidazolinyl, pyrrolidinyl, piperidinyl, piperazinyl, morpholinyl, thiomorpholinyl, tetrahydropyranyl and tetrahydrofuranyl, wherein said heterocyclic group is optionally mono- or polysubstituted with:
(a) alkyl of 1 to 3 carbon atoms,
(b) —COOH,
(ci) a group of the formula —C(O)R⁶, wherein R⁶ is straight or branched alkyl of 1 to 5 carbon atoms or cycloalkyl of 3 to 5 carbon atoms,
(g) a group of the formula —NR⁷R⁸, wherein R⁷ and R⁸ are each independently a hydrogen atom or a methyl group,
(h) a group of the formula —CONR⁹R¹⁰, wherein R⁹ and R¹⁰ are each independently a hydrogen atom or a methyl group,
(i) a group of the formula —OR¹¹, wherein R¹¹ is a hydrogen atom, or an alkyl or acyl group of 1 to 7 carbon atoms,
(g) —CN, or
(h) halogen,
R² is H; or
R¹ and R² together with the nitrogen they are bonded to may form a heterocyclic ring selected from the class consisting of piperidine, pyrrolidine, piperazine and morpholine, wherein said heterocyclic ring is optionally mono-, di- or trisubstituted with:
(a) alkyl of 1 to 3 carbon atoms,
(b) —COOH,
(g) a group of the formula —C(O)R⁶, wherein R⁶ is straight or branched alkyl of 1 to 5 carbon atoms or cycloalkyl of 3 to 5 carbon atoms,
(h) a group of the formula —NR⁷R⁸, wherein R⁷ and R⁸ are each independently a hydrogen atom or a methyl group
(i) a group of the formula —CONR⁹R¹⁰, wherein R⁹ and R¹⁰ are each independently a hydrogen atom or a methyl group,
(j) a group of the formula —OR¹¹, wherein R¹¹ is a hydrogen atom, or an alkyl or acyl group of 1 to 7 carbon atoms,
(h) —CN,
(i) halogen,
R³ is
phenyl, which is optionally substituted in the 4-position with:
(A) R⁴⁴, which is aryl or heteroaryl selected from the class consisting of phenyl, thiophenyl, pyridyl, pyrimidinyl and furyl, wherein one or more of the hydrogen atoms of said aryl or heteroaryl group is optionally and independently replaced with:
(i) branched or unbranched alkyl of 1 to 6 carbon atoms or cycloalkyl of 3 to 6 carbon atoms, which alkyl or cycloakyl group is optionally mono- or polysubstituted with halogen or oxo,
(ii) —CN,
(iii) nitro, or
(iv) halogen,
(D) methyl,
(E) branched or unbranched alkyl of 2 to 6 carbon atoms or cycloalkyl of 3 to 6 carbon atoms, which alkyl or cycloakyl group is optionally monosubstituted with halogen or oxo,
(D) a group of the formula —COOR⁵², wherein R⁵² is straight or branched alkyl of 1 to 5 carbon atoms or cycloalkyl of 3 to 5 carbon atoms,
(E) a group of the formula —COR⁵⁷, wherein R⁵⁷ is a hydrogen atom, straight or branched alkyl of 1 to 5 carbon atoms, cycloalkyl of 3 to 5 carbon atoms,
(F) a group of the formula —OR⁵⁸, wherein R⁵⁸ is a hydrogen atom, an alkyl, fluoroalkyl or acyl group of 1 to 7 carbon atoms,
(G) —CN,
(H) nitro, or
(I) halogen;
R³ is Cl;
X is ═C(H)—; and,
R⁴ is Cl;
or a pharmaceutically acceptable salt thereof.

4. A compound of the formula I, as set forth in claim 1, wherein:
Y is an oxygen atom;
Z is an oxygen atom;
R¹ is selected from the class consisting of:
branched or unbranched alkyl of 1 to 10 carbon atoms or alkenyl of 2 to 10 carbon atoms, wherein one to three carbon atoms in said alkyl, alkenyl, cycloalkyl or cycloalkenyl may be replaced by —O—, —NH— or —N(CH₃)—, and in which said alkyl or alkenyl group one to six hydrogen atoms are optionally and independently replaced with:
(i) halogen,
(ii) oxo,
(viii) aryl or heteroaryl which is selected from the class consisting of phenyl, indolyl, pyridyl, pyrimidinyl, oxazolyl, thiazolyl, pyrazolyl, imidazolyl, pyrazinyl, benzothiazolyl and benzimidazolyl, wherein one or more hydrogen atoms of said aryl or heteroaryl group are optionally and independently replaced with:
(a) alkyl of 1 to 3 carbon atoms,
(b) —COOH,
(ci) a group of the formula —NR⁷R⁸, wherein R⁷ and R⁸ are each independently a hydrogen atom or a methyl group,
(di) a group of the formula —OR¹¹, wherein R¹¹ is a hydrogen atom or a methyl group,
(e) —CN,
(i) halogen,
(iv) —CN,
(v) a group of the formula —OR¹⁹, wherein R¹⁹ is a hydrogen atom, a methyl group or an acetyl group,
(vi) a group of the formula —NR²¹R²², wherein R²¹ and R²² are each, independently,
(a) a hydrogen atom,
(b) methyl
(vii) a saturated, or partially unsaturated heterocyclic group selected from piperidinyl, piperazinyl and morpholinyl, wherein said heterocyclic group is optionally mono- or polysubstituted with:
(a) alkyl of 1 to 3 carbon atoms,
(b) —COOH,
(ci) a group of the formula —C(O)R⁶, wherein R⁶ is straight or branched alkyl of 1 to 5 carbon atoms or cycloalkyl of 3 to 5 carbon atoms,
(di) a group of the formula —OR¹¹, wherein R¹¹ is a hydrogen atom, or an alkyl or acyl group of 1 to 7 carbon atoms, or
(e) halogen,
R² is H; or
R¹ and R² together with the nitrogen they are bonded to may form a heterocyclic ring selected from the class consisting of piperidine, pyrrolidine, piperazine and morpholine, wherein said heterocyclic ring is optionally mono-, di- or trisubstituted with:
(a) alkyl of 1 to 3 carbon atoms,
(b) —COOH,
(ci) a group of the formula —C(O)R⁶, wherein R⁶ is straight or branched alkyl of 1 to 5 carbon atoms or cycloalkyl of 3 to 5 carbon atoms,
(di) a group of the formula —NR⁷R⁸, wherein R⁷ and R⁸ are each independently a hydrogen atom or a methyl group, or
(f) a group of the formula —OR¹¹, wherein R¹¹ is a hydrogen atom, or an alkyl or acyl group of 1 to 7 carbon atoms,
R³ is
phenyl, which is optionally substituted in the 4-position with:
(F) R⁴⁴, which is aryl or heteroaryl selected from the class consisting of phenyl, pyridyl and pyrimidinyl,
(G) methyl,
(H) CN
(I) nitro, or
(J) halogen;
R³ is Cl;
X is ═C(H); and,
R⁴ is Cl;
or a pharmaceutically acceptable salt thereof.

5. A compound of the formula I, as set forth in claim 1, wherein:
Y is an oxygen atom;
Z is an oxygen atom;
R¹ is selected from the class consisting of:
branched or unbranched alkyl of 1 to 8 carbon atoms or alkenyl of 2 to 8 carbon atoms, wherein one to three carbon atoms in said alkyl, alkenyl, cycloalkyl or cycloalkenyl may be replaced by —O—, and in which said alkyl or alkenyl group one to six hydrogen atoms are optionally and independently replaced with:
(i) oxo,
(iii) aryl or heteroaryl which is selected from the class consisting of phenyl, pyridyl and imidazolyl, wherein one or more hydrogen atoms of said aryl or heteroaryl group are optionally and independently replaced with:
(a) methyl,
(b) —COOH,
(ci) a group of the formula —OR¹¹, wherein R¹¹ is a hydrogen atom or a methyl group,
(iii) a group of the formula —OR¹⁹, wherein R¹⁹ is a hydrogen atom, a methyl group or an acetyl group,
R² is H; or
R¹ and R² together with the nitrogen they are bonded to may form a heterocyclic ring selected from the class consisting of piperidine, pyrrolidine, piperazine and morpholine, wherein said heterocyclic ring is optionally mono-, di- or trisubstituted with:
(a) alkyl of 1 to 3 carbon atoms,
(b) —COOH,
(ci) a group of the formula —C(O)R⁶, wherein R⁶ is straight or branched alkyl of 1 to 5 carbon atoms or cycloalkyl of 3 to 5 carbon atoms,
(di) a group of the formula —OR¹¹, wherein R¹¹ is a hydrogen atom, a methyl group or an acetyl group,
R³ is
phenyl, which is optionally substituted in the 4-position with:
(F) R⁴⁴, which is aryl or heteroaryl selected from the class consisting of phenyl, pyridyl and pyrimidinyl,
(G) methyl,
(H) CN
(I) nitro, or
(J) halogen;
R⁴ is Cl;
X is ═C(H); and,
R⁵ is Cl;
or a pharmaceutically acceptable salt thereof.

6. A compound of the formula I, in accordance with claim 1, 2, 3,4, 5, or 6, with the absolute stereochemistry depicted below in formula II (below).

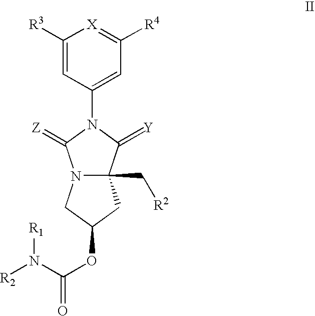

7. A compound selected from the group consisting of: 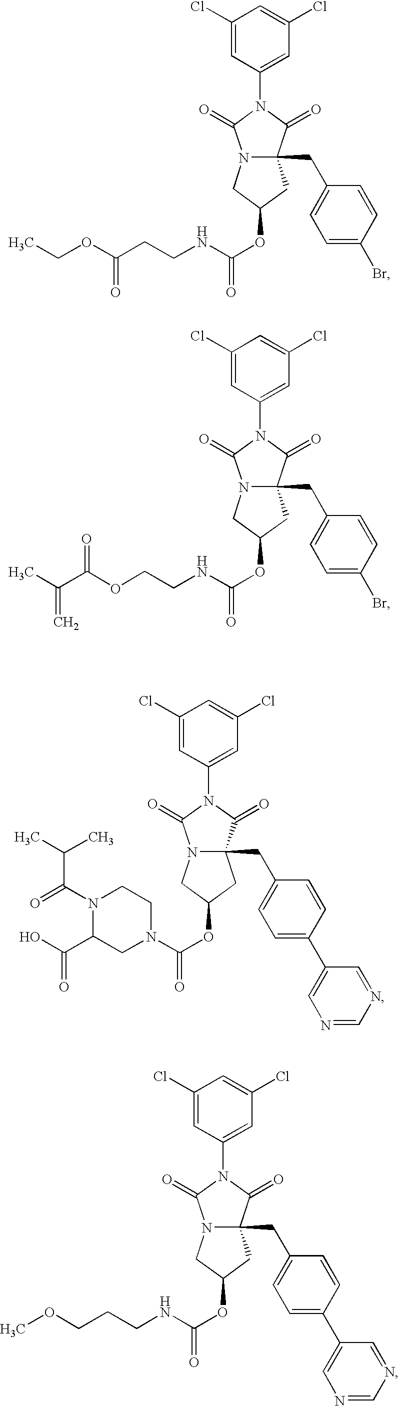 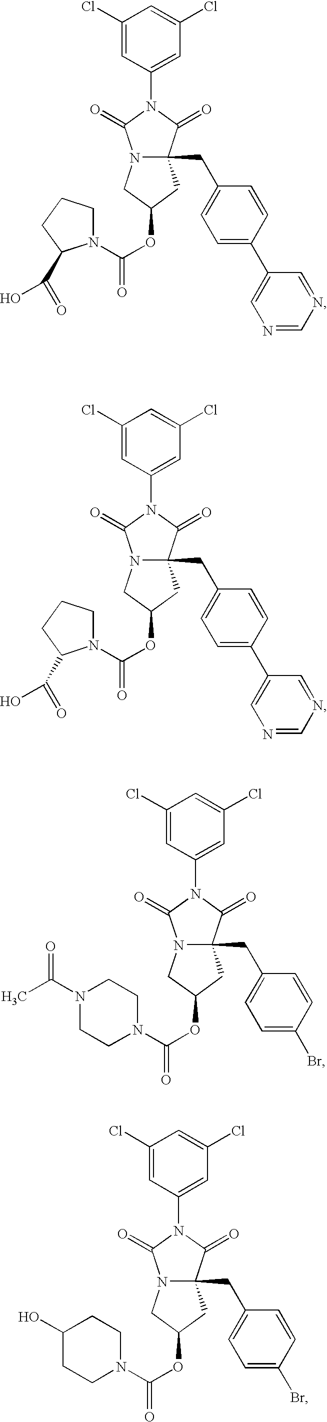 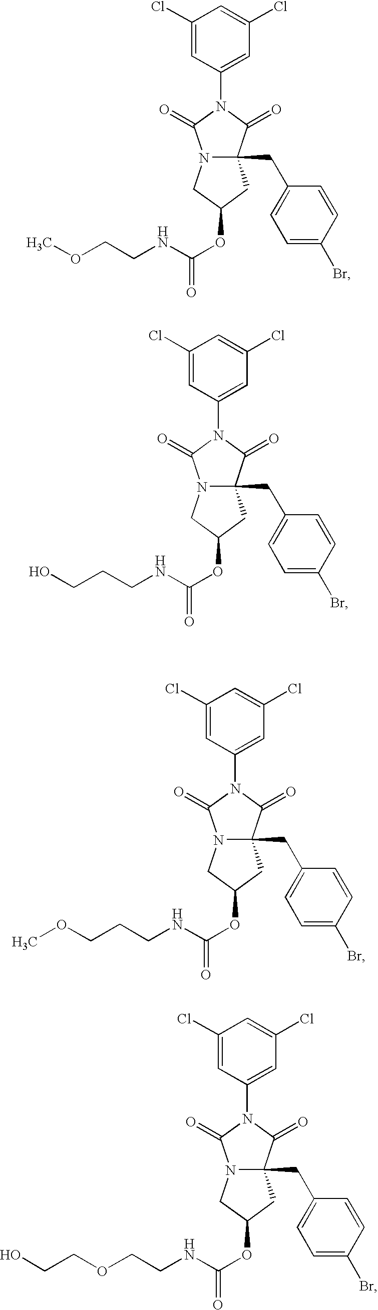 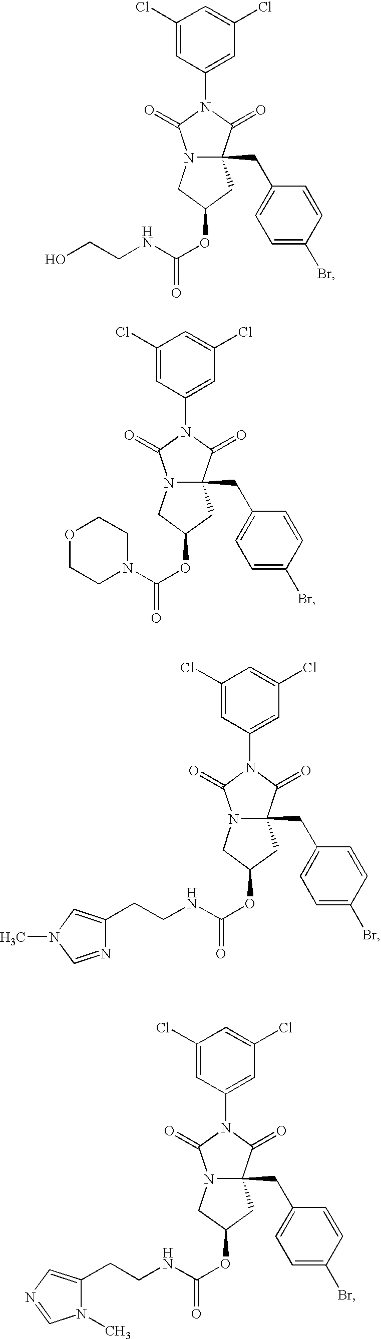 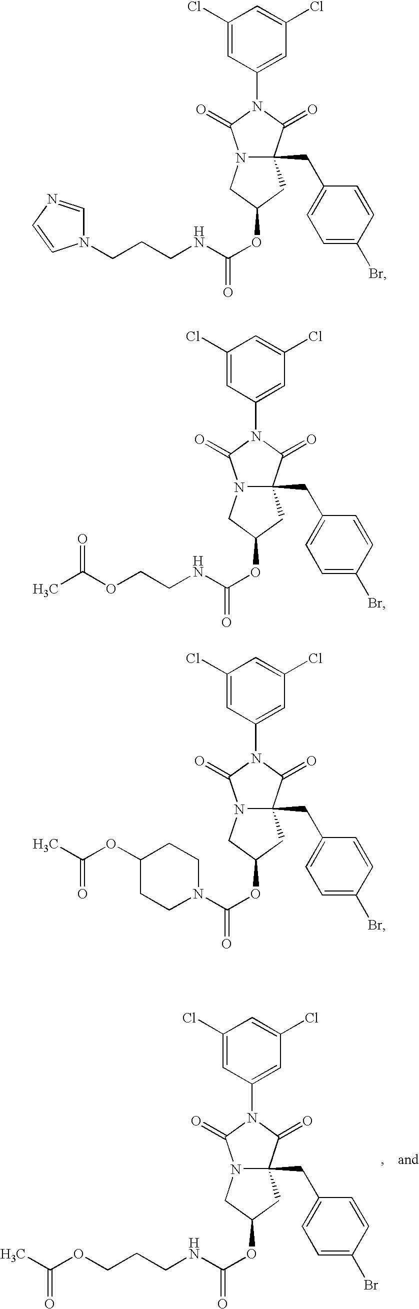 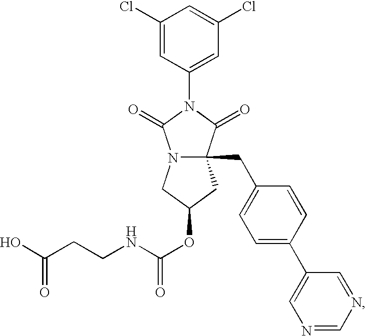
or a pharmaceutically acceptable salt thereof.

8. A method for the treatment of inflammatory or immune cell-mediated diseases which comprises administering to a host in need or such treatment or prophylaxis a therapeutic amount of a compound in accordance with claim 1, 2, 3, 4, 5, 6 or 7.

9. The method of claim 8 wherein the disease or condition is selected from the group consisting of adult respiratory distress syndrome, shock, oxygen toxicity, multiple organ injury syndrome secondary to septicemia, multiple organ injury syndrome secondary to trauma, reperfusion injury of tissue due to cardiopulmonary bypass, myocardial infarction or use with thrombolysis agents, acute glomerulonephritis, vasculitis, reactive arthritis, dermatosis with acute inflammatory components, stroke, thermal injury, hemodialysis, leukapheresis, ulcerative colitis, necrotizing enterocolitis and granulocyte transfusion associated syndrome.

10. The method of claim 8 wherein the disease or condition is selected from the group consisting of psoriasis, organ/tissue transplant rejection, graft vs. host reactions and autoimmune diseases including Raynaud's syndrome, autoimmune thyroiditis, dermatitis, multiple sclerosis, rheumatoid arthritis, insulin-dependent diabetes mellitus, uveitis, inflammatory bowel disease including Crohn's disease and ulcerative colitis; and systemic lupus erythematosus.

11. The method of claim 8 wherein the disease or condition is asthma.

12. The method of claim 8 wherein the condition is toxicity associated with cytokine therapy.

13. The method of claim 8 wherein the disease or condition is psoriasis.

14. A pharmaceutical composition comprising a compound in accordance with claim 1, 2, 3, 4, 5, 6 or 7 and a pharmaceutically acceptable carrier or diluent.
